(19) United States
(12) Patent Application Publication
(10) Pub. No.: US 2024/0192213 A1
(43) Pub. Date: Jun. 13, 2024

(54) METHOD OF DETECTING DISEASE RELATED BIOMARKERS IN BODILY FLUID SAMPLE

(71) Applicant: The Administrators of the Tulane Educational Fund, New Orleans, LA (US)

(72) Inventors: Ye Tony HU, New Orleans, LA (US); Bo NING, New Orleans, LA (US); Yating XIAO, New Orleans, LA (US)

(21) Appl. No.: 18/556,344

(22) PCT Filed: Apr. 20, 2022
(86) PCT No.: PCT/US22/25608

§ 371 Date: Oct. 19, 2023

Related U.S. Application Data
(60) Provisional application No. 63/176,986, filed on Apr. 20, 2021.

(51) Int. Cl. G01N 33/569; C12Q 1/6851; C12Q 1/70; C12Q 1/6804

(52) U.S. Cl. CPC G01N 33/56983; C12Q 1/6851; C12Q 1/701; C12Q 1/6804; G01N 2333/165; G01N 2469/10; G01N 2469/20

(57) ABSTRACT

Method and system for detecting disease in blood sample is described. By extracting extracellular vesicles (EVs) from the blood sample and employing liposome fusion probes, it is shown that the test can be completed within hours instead of weeks, and the detection limit can be significantly lowered.

### PRIOR RELATED APPLICATIONS

This application claims priority to U.S. Ser. No. 63/176,986, filed Apr. 20, 2021, which is incorporated by reference herein in its entirety for all purposes.

### FEDERALLY SPONSORED RESEARCH STATEMENT

This study was supported by grants R01AI113725, R01AI122932, 1R03AI140977-01 and R21A1126361 from the National Institute of Allergy and Infectious Diseases (NIAID), R01HD090927 from the National Institute of Child Health and Human Development, 1U01CA252965-01 from the National Cancer Institute and W8IXWH1910926 from the Department of Defense, NIAID Contract No. HHSN272201700033I and grant OD011104 from the NIH Office of Research Infrastructure Programs.

### FIELD OF THE DISCLOSURE

The disclosure generally relates to a method for diagnosis of disease in a blood sample, and more particularly relates to a method for detecting disease-derived antigen on circulating extracellular vesicles (EVs) in the blood sample by RNA detection and amplification methods, such as CRISPR/Cas-12.

### BACKGROUND OF THE DISCLOSURE

Tremendous need exists for better diagnostic methods in various disease states, including cancer, viral infections and bacterially induced illness. For example, in the recent experience with SARS-CoV-2, reverse-transcriptase quantitative polymerase chain reaction (RT-qPCR) analysis of respiratory samples has been the gold-standard for COVID-19 diagnosis, but has limitations. SARS-CoV-2 RNA levels in the upper respiratory tract rapidly decrease after infection while lower respiratory tract levels remain high. RT-qPCR assays performed after early SARS-CoV-2 infection may thus yield false-negatives, but infection events are often unclear, complicating interpretation. Nasopharyngeal tissue highly expresses ACE2, the primary receptor for SARS-CoV-2, but ACE2 is expressed in other tissues (e.g., cardiac and small intestine) reported to develop SARS-CoV-2 infections and related pathology. Gold-standard nasopharyngeal RT-qPCR results thus may not accurately reflect the status of lower respiratory tract or extrapulmonary infections.

Circulating SARS-CoV-2 RNA detectable in mild to severe COVID-19 cases correlates with and predicts COVID-19 disease severity, and appears responsible for extrapulmonary infections. RT-qPCR exhibits poor overall sensitivity when applied to detect SARS-CoV-2 RNA in plasma (≤41%), however, and no reports appear to address high-sensitivity detection of SARS-CoV-2 RNA in serum or plasma, with the exception of two studies employing droplet-based digital RT-PCR, which is not suitable for clinical applications. More sensitive and robust blood-based SARS-COV-2 RNA detection methods are needed to improve the diagnosis and prognostic evaluation of COVID-19 cases, and should be compatible with routine clinical tests to simplify sample collection.

Notably, infected cells may abundantly secrete EVs containing pathogen-derived factors, which can accumulate in the circulation while protecting their contents from environmental hydrolases. Hepatitis A and C are also reported able to infect cells by EV-mediated transfer of their viral genomes, suggesting that SARS-CoV-2, which employs the same genome structure, could utilize a similar mechanism. Such virus-loaded EVs could serve as indicators of systemic viral load and disease severity, but most EV isolation methods are not feasible as clinical applications.

The development of an assay where EVs are directly captured from plasma using an antibody to an EV surface protein, and then fused with liposomes containing RT, recombinase polymerase amplification (RPA), and CRISPR/Cas12a reagents to produce a clinically viable workflow similar to diagnostic ELISAs would address diagnostic need across many disease states (FIG. 1). In such an assay, guide-RNA directs CRISPR/Cas12a binding to an RT-RPA target amplicon, inducing concentration dependent cleavage of a quenched oligonucleotide probe for ultrasensitive detection of SARS CoV-2 RNA (FIG. 2). This approach employs two mature technologies: antibody mediated target capture and liposome-mediated reagent delivery, while extensive literature describes liposome synthesis and stabilization for clinical applications.

Conditions have been optimized for a one-step CRISPR-enhanced RT-RPA fluorescent detection system (CRISPR-FDS) suitable for incorporation into liposome fusion probes, using assay conditions from a previous study as the starting point. RT-RPA reactions performed at room temperature (˜22° C.) did not reveal marked CRISPR-mediated probe conversion under optimal CRISPR conditions, while RT-RPA reactions performed within the optimal RT-RPA temperature range produced similar CRISPR-FDS signal (FIG. 3). CRISPR reactions performed with a constant amount of amplicon demonstrated different reaction kinetics but similar endpoints, with reactions performed at 22° C., 37° C. and 42° C. reaching their respective maximum signal intensities at approximately 25, 13 and 10 minutes (FIG. 4). CRISPR-FDS assays using these optimized conditions to analyze plasma EVs RNA isolates accurately distinguished individuals with and without COVID-19 (FIG. 5). However, negative control samples containing genomic RNA from other human respiratory viruses, including influenza and several alpha and beta coronaviruses (FIGS. 6, 7), produced no signal, confirming the SARS-CoV-2-specificity of this one-step assay approach (FIG. 8).

An ideal invasive test should accurately measure any quantity of host targets in body fluids to achieve the highest accuracy for diseases at different phases, including latent infection. Extracellular vesicles (EVs) that are heavily implicated in pathogenic process could contain many targets for marker discovery.

Therefore, there is a need to develop assays to enable rapid, quantitative, ultrasensitive testing methods for detecting various diseases.

### SUMMARY OF THE DISCLOSURE

In one aspect of this disclosure, a method of detecting a disease-specific protein in a bodily fluid sample is described. The method comprises the steps of: (a) extracting extracellular vesicles (EVs) in the bodily fluid sample by use of a first antibody against the disease-specific protein; (b) mixing liposome fusion probes with the EVs in step (a), wherein the liposome fusion probes contain RT, recombinase polymerase amplification (RPA), and CRISPR/Cas12a reagents; and (c) detecting the presence of the disease-specific protein using a fluorescent detection system.

In one embodiment, the bodily fluid sample can be obtained from a patient. For example, the bodily fluid can be blood, serum, sputum, urine, or other available bodily fluid.

In one embodiment, the EVs are extracted by using a capture antibody such as anti-CD81 antibodies. Detection antibodies that recognizes surface markers on exosome or disease-derived EVs can also be used, and non-limiting examples include CD4, CD8, CD9, CD19, CD20, CD57, CD91, CD63, PDCD6IP, HSPA8, PD-1, PD-L1, TSPN8, EGFR, HER2, KRAS, ACE2, TMEM119, ANXA2, ANXA5, HSP90AB1, YWHAZ, YWHAE, LprG, LpqH, LAM, Ag85B, EpCAM, EphA2, Alpha-crystallin (HspX), DnaK, GroEL2, KatG, SodA and GlnA, GP120, GP160, GP40 etc.

In another aspect of this disclosure, a method of detecting the presence of a disease-specific protein in a blood sample is described. The method comprises the steps of: (a) extracting extracellular vesicles (EVs) in the blood sample by use of a first antibody against the disease-specific protein; (b) mixing liposome fusion probes with the mixture, wherein the liposome fusion probes contain RT, recombinase polymerase amplification (RPA), and CRISPR/Cas12a reagents; and (c) detecting the presence of the disease-specific protein using fluorescent detection system.

As used herein, “sample” refers to a small amount of biological substance collected from a person to be examined.

In one embodiment, “Disease” is SARS-CoV-2 infection, however, a broad range of diseases can be targeted, as long as nucleic acid biomarkers can be detected in EV, including but not limited to: infectious diseases such as TB, HIV, or influenza; cancers such as lung cancer, breast cancer, pancreatic cancer, leukemia, or lymphoma; and brain damage or neuron degeneration.

As used herein, “RNA” refers to nucleic acid targets such as RNA from pathogens virus or bacteria or human messenger RNA (mRNA), non-coding RNA (ncRNA), micro-RNA (miRNA). Human circulating DNA and pathogen DNA are also can be target using such detection system.

In one embodiment, nucleic acid target can be in mutation form, such as D614G mutation in SARS-CoV-2 viral RNA, Kras G12C, G12D and G12R mutation in human cancer.

As used herein, “RT-RPA” refers but not limited to reverse transcription and recombinase polymerase amplification (RPA) reaction. Any nucleic acid amplification method applied to this method, such as PCR, RT-PCR, LAMP, RT-LAMP, RCA, EXPAR, WGA, SDA, HAD, NASBA etc.

In one embodiment, CRISPR effector protein is selected from a group consisting of Cas12a, Cas9 and Cas13. In one embodiment, the CRISPR effector protein is Cas12a. However, other CRISPR effector proteins can be used, as long as effective detection with high specificity can be achieved.

As used herein, “CRISPR proteins” or “CRISPR effector protein” or “CRISPR enzymes” refers to Class 2 CRISPR effector proteins including but not limited to Cas9, Cas12a (formerly known as Cpf1), Csn2, Cas4, C2c1, Cc3, Cas13a, Cas13b, Cas13c, Cas13d. In one embodiment, the CRISPR effector proteins described herein are preferably Cpf1 effector proteins.

As used herein, “guide RNA” or “gRNA” refers to the non-coding RNA sequence that binds to the complementary target DNA sequence to guide the CRISPR-Cas system in close contact with the target DNA strand.

As used herein, a “reporter molecule” refers to a single-stranded DNA or single-stranded RNA that is labeled with fluorescence and quencher, gold nanoparticles or biotin-FAM, and the dissociation of the reporter can be detected by either a fluorescence reader or colorimetric change.

As used herein, “extracellular vesicles” or “EV” refers to lipid bilayer-delimited particles that are naturally released from a cell, bacterial and cannot replicate themselves. EVs range in diameter from about 20-30 nm to about 10 μm or more. EVs are capable of transferring nucleic acids, such as RNA, between cells. EVs are typically separated from a blood sample by ultracentrifuge or density gradient ultracentrifugation, size exclusion chromatography, ultrafiltration, and affinity/immunoaffinity capture method. There are certain EV-enriched markers that can be used to better isolate EVs. Examples of EV-enriched markers include, but not limited to, CD4, CD8, CD9, CD19, CD20, CD57, CD91, CD63, PDCD6IP, HSPA8, PD-1, PD-L1, TSPN8, EGFR, HER2, KRAS, ACE2, TMEM119, ANXA2, ANXA5, HSP90AB1, YWHAZ, YWHAE, LprG, LpqH, LAM, Ag85B, EpCAM, EphA2, Alpha-crystallin (HspX), DnaK, GroEL2, KatG, SodA and GlnA, GP120, GP160, GP40 etc.

As used herein, “fluorescent detection system” refers to a process in which light from an excitation source passes through a filter or monochromator, and strikes a sample. A proportion of the incident light is absorbed by the sample, and some of the molecules in the sample fluoresce. The fluorescent light is emitted in all directions. Some of this fluorescent light passes through a second filter or monochromator and reaches a detector, which is usually placed at 90° to the incident light beam to minimize the risk of transmitted or reflected incident light reaching the detector.

As used here, “liposome fusion probes” refers to nanoscale liposomes synthesized to deliver RT-RPA-CRISPR reagents to captured EVs.

As used herein, “liposome” refers to but not limited to nanoscale a spherical vesicle having at least one lipid bilayer synthesized with 1,2-Dimyristoyl-sn-glycerol-3-phosphorylcholine (DMPC) and cholesterol. Liposome also can be produced by cell membrane from cell lines and primary cells.

As used herein, “fusion” refers to the process by which two initially distinct lipid bilayers merge their hydrophobic cores, resulting in one interconnected structure. The process used herein can be mediated but not limited to Polyethylene glycol 8000.

In another aspect of this disclosure, a non-immobilized method of process liposome and EV is can be applied. Antibody against disease biomarkers can be conjugated into liposome surface by syntheses.

As used herein, “LAM” refers to lipoarabinomannan in MTBs. LAM antigen, present in mycobacterial cell walls, which is released from metabolically active or degenerating bacterial cells. LAM appears to be present predominately in people with active TB disease.

As used herein, “Ag85B” refers to antigen 85B found in MTB, which is a fibronectin-binding protein with mycolyltransferase activity, is the major secretory protein in actively replicating MTB.

As used herein, “LpqH” refers to Lipoprotein LpqH found in MTBs. The 19 kDa Mycobacterium tuberculosis lipoprotein (LpqH) induces macrophage apoptosis through extrinsic and intrinsic pathways: a role for the mitochondrial apoptosis-inducing factor.

As used herein, “alpha-crystallin (HspX)” refers to a 16 kDa heat shock protein HspX that is required for mycobacterium persistence within microphages.

As used herein, “DnaK” refers to bacterial molecular Chaperone protein DnaK. Chaperones are proteins that bind to other proteins, thereby stabilizing them in an ATP-dependent manner. DnaK is an enzyme that couples cycles of ATP binding, hydrolysis, and ADP release by an N-terminal ATP-hydrolysing domain to cycles of sequestration and release of unfolded proteins by a C-terminal substrate binding domain.

As used herein, “GroEL2” refers to the 60 kDa chaperonin 2 (aka Cpn60.2) that is closely related to Cpn60.1 chaperone localized within the outer layer of M. tuberculosis cell wall. GroEL2 is found to be present in the cerebrospinal fluid of TB meningitis patients.

As used herein, “KatG” refers to Catalase-peroxidase, which activates the pro-drug INH that is coded by the katG gene in M. tuberculosis. Mutations of the katG gene in M. tuberculosis are a major INH resistance mechanism.

As used herein, “SodA” refers to Superoxide dismutase [Fe]. For MTB detection purposes, unless otherwise specified, SodA refers particularly to MTB SodA.

As used herein, “GlnA” refers to Glutamine synthetase. For MTB detection purposes, GlnA refers particularly to MTB GlnA.

As used herein, “PDCD6IP” refers to programmed cell death 6-interacting protein, which encodes a protein thought to participate in programmed cell death.

As used herein, “HSPA8” refers to human heat shock 70 kDa protein 8, also known as heat shock cognate 71 kDa protein or Hsc70 or Hsp73. As a member of the heat shock protein 70 family and a chaperone protein, it facilitates the proper folding of newly translated and misfolded proteins, as well as stabilizing or degrading mutant proteins.

As used herein, “CD4” refers to human CD4, which is a glycoprotein found on the surface of immune cells such as T helper cells, monocytes, macrophages, and dendritic cells that have been identified in humans.

As used herein, “CD8” refers to human CD8, which is a transmembrane glycoprotein that serves as a co-receptor for the T-cell receptor. Along with the TCR, the CD8 co-receptor plays a role in T cell signaling and aiding with cytotoxic T cell antigen interactions.

As used herein, “CD16” refers to human CD16, which is also known as FcγRIII, is a cluster of differentiation molecule found on the surface of natural killer cells, neutrophils, monocytes, and macrophages.

As used herein, “CD19” refers to human CD19, which is also known as CD19 molecule, B-Lymphocyte Surface Antigen B4, T-Cell Surface Antigen Leu-12 and CVID3 is a transmembrane protein that in humans is encoded by the gene CD19. In humans, CD19 is expressed in all B lineage cells.

As used herein, “CD20” refers to human CD20, which is also known as B-lymphocyte antigen CD20 or CD20 is expressed on the surface of all B-cells beginning at the pro-B phase and progressively increasing in concentration until maturity.

As used herein, “CD57” refers to human CD57, which is 3-beta-glucuronosyltransferase 1 is an enzyme that in humans is encoded by the B3GAT1 gene, whose enzymatic activity creates the CD57 epitope on other cell surface proteins. In immunology, the CD57 antigen is also known as HNK1 or LEU7.

As used herein, “PD1” refers to human Programmed cell death protein 1, which is an inhibitory receptor that is expressed by all T cells during activation. It regulates T cell effector functions during various physiological responses, including acute and chronic infection, cancer and autoimmunity, and in immune homeostasis.

As used herein, “PDL1” refers to human Programmed death-ligand 1, which is also known as cluster of differentiation 274 or B7 homolog 1 is a protein that in humans is encoded by the CD274 gene

As used herein, “EGFR” refers to human epidermal growth factor receptor, which is a protein present on the surface of both healthy cells and cancer cells. When damaged, as can occur in some lung cancer cells, EGFR doesn't perform the way it should. Instead, it causes rapid cell growth, helping the cancer spread.

As used herein, “HER2” refers to human Receptor tyrosine-protein kinase erbB-2, which is also known as CD340, proto-oncogene Neu, Erbb2, or ERBB2, is a protein that in humans is encoded by the ERBB2 gene. ERBB is abbreviated from erythroblastic oncogene B, a gene isolated from avian genome. It is also frequently called HER2 or HER2/neu.

As used herein, “KRAS” refers to human protein called K-Ras, part of the RAS/MAPK pathway, The protein relays signals from outside the cell to the cell's nucleus. These signals instruct the cell to grow and divide (proliferate) or to mature and take on specialized functions (differentiate).

As used herein, “TSPAN8” refers to human Tetraspanin 8, which is a protein that in humans is encoded by the TSPAN8 gene and reported associated with long cancer.

As used herein, “EpCAM” refers to human Epithelial cell adhesion molecule, which is a transmembrane glycoprotein mediating Ca²⁺-independent homotypic cell-cell adhesion in epithelia. EpCAM is also involved in cell signaling, migration, proliferation, and differentiation.

As used herein, “EphA2” refers to human ephrin type-A receptor 2, which is a transmembrane glycoprotein composed of 976 amino acid residues, with a calculated molecular mass of 130 kDa.

As used herein, “ACE2” refers to human Angiotensin-converting enzyme 2, which is an enzyme attached to the cell membranes of cells located in the lungs, arteries, heart, kidney, and intestines.

As used herein, “TEME119” refers to human Transmembrane Protein 119, which is specifically expressed by parenchymal myeloid cells in the central neuron system. TMEM119 is known as a microglia-specific and robustly expressed trans-membranous molecule

As used herein, “GP120” refers to HIV Envelope glycoprotein GP120, which is a glycoprotein exposed on the surface of the HIV envelope. The 120 in its name comes from its molecular weight of 120 kDa.

As used herein, “GP41” refers to HIV Envelope glycoprotein GP141, which is also known as glycoprotein 41 is a subunit of the envelope protein complex of retroviruses, including human immunodeficiency virus (HIV). Gp41 is a transmembrane protein that contains several sites within its ectodomain that are required for infection of host cells.

As used herein, “GP160” refers to the envelope glycoprotein of human immunodeficiency virus type 1, envelope glycoprotein is synthesized as a precursor glycoprotein, gp160, and is then processed into gp120 and gp41.

As used herein, “ACTB” refers to human beta-actin, which is one of six different actin isoforms that have been identified in humans.

As used herein, “ANXA2” refers to annexin A2, which is involved in diverse cellular processes such as cell motility, linkage of membrane-associated protein complexes to the actin cytoskeleton, endocytosis, fibrinolysis, ion channel formation, and cell matrix interaction.

As used herein, “PKM” refers to pyruvate kinase M1/2, which catalyzes the transfer of a phosphoryl group from phosphoenolpyruvate to ADP, generating ATP and pyruvate.

As used herein, “HSP90AA1” refers to human heat shock protein HSP 90-alpha (cytosolic), member A1. Complemented by the constitutively expressed paralog Hsp90B which shares over 85% amino acid sequence identity, Hsp90A expression is initiated when a cell experiences proteotoxic stress. Once expressed Hsp90A dimers operate as molecular chaperones that bind and fold other proteins into their functional 3-dimensional structures.

As used herein, “ENO1” refers to alpha-enolase, which is a glycolytic enzyme expressed in most tissues. Each isoenzyme is a homodimer composed of 2 alpha, 2 gamma, or 2 beta subunits, and functions as a glycolytic enzyme. Alpha-enolase, in addition, functions as a structural lens protein (tau-crystallin) in the monomeric form.

As used herein, “ANXA5” refers to annexin A5, which is a cellular protein in the annexin group. ANXA5 is able to bind to phosphatidylserine, a marker of apoptosis when it is on the outer leaflet of the plasma membrane.

As used herein, “HSP90AB1” refers to heat shock protein HSP 90-beta, a molecular chaperone.

As used herein, “YWHAZ” refers to 14-3-3 protein zeta/delta, which is a member of the 14-3-3 protein family and a central hub protein for many signal transduction pathways. It is a major regulator of apoptotic pathways critical to cell survival and plays a key role in a number of cancers and neurodegenerative diseases.

As used herein, “YWHAE” refers to 14-3-3-protein epsilon, a member of the 14-3-3 family that mediate signal transduction by binding to phosphoserine-containing proteins.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| Ag85b | Antigen 85B |
| AuNRs | Gold Nanorods |
| EVs | Extracellular Vesicles or exosomes |
| LAM | lipoarabinomannan |
| LC | Liquid chromatography |
| MS | mass spectrometry |
| MTB | Mycobacterium tuberculosis |
| nPES | Nanoplasmon-enhanced scattering |
| RPA | recombinase polymerase amplification |
| RT | Reverse-transcription |
| RT-qPCR | Reverse-transcriptase quantitative polymerase chain |
| | reaction |
| TB | Tuberculosis |
| CRISPR | Clustered regularly interspaced short palindromic |
| | repeats |
| gRNA | Guide RNA |
| LAMP | Loop-mediated isothermal amplification |
| NASBA | Nucleic acid sequence-based amplification |
| PAM | Protospacer adjacent motif |
| PCR | Polymerase chain reaction |
| RCA | Rolling circle amplification |
| WGA | Whole Genome Amplification |
| SDA | Strand Displacement Amplification |
| HDA | Helicase-Dependent Amplification |

### BRIEF DESCRIPTION OF THE DRAWINGS

FIG. 1. Schematic of the proposed assay, indicating CD81-mediated capture of plasma EVs, their fusion with RT-RPA-CRISPR-loaded liposomes, RT-RPA-mediated target amplification, and signal generation by CRISPR-mediated cleavage of a quenched fluorescent probe in proportion to target amplificon concentration. Analysis sample types include cell culture media and plasma from non-human primate (NHP) COVID-19 disease models and COVID-19 patients.

FIG. 2. Detection mechanism and workflow of CRISPR-based RNA detection. CRISPR-FDS assays utilize simultaneous isothermal reverse transcriptase and RPA reactions to amplify a target region that is then transiently bound in a sequence-specific manner by a CRISPR-Cas12a guide RNA (gRNA) complex, with binding specificity determined by the gRNA sequence specificity (e.g., SARS-CoV-2 N gene). Cas12a/gRNA binding activates this enzyme complex to rapidly and nonspecifically cleave an interacting single-stranded poly T DNA oligonucleotide probe present in large molar excess. Cleavage of the assay probe unmasks its quenched fluorescent label to produce fluorescent in proportion to amount of available amplicon in the reaction, which directly reflects the amount of SARS-CoV-2 RNA present in the analysis sample. CRISPR-FDS fluorescent signal development is rapid since probe cleavage occurs in parallel with the amplification of its sequence target, producing a fluorescent signal that can be sensitively read by benchtop plate reader or cellphone-based chip reader, and compared to negative and positive control (NC and PC) samples and concentration standards to detect and quantify the amount of SARS-CoV-2 RNA present in the analyzed sample.

FIG. 3. Optimization of RT-RPA-CRISPR assay conditions. CRISPR-FDS assay signal from 5 μL PBS aliquots spiked with 100 copies of SARS-CoV-2 RNA and then incubated at 22˜42° C. for 15 min with 15 μL of RT-RPA reagents and at 37° C. for 15 min with 50 μL of CRISPR reagents.

FIG. 4. Optimization of RT-RPA-CRISPR assay conditions. CRISPR-FDS assay signal from 5 μL PBS aliquots spiked with 100 copies of SARS-CoV-2 RNA and then incubated at 37° C. for 15 min with 15 μL of RT-RPA reagents and at 22-42° C. 15 min with 50 μL of CRISPR reagents.

FIG. 5. Optimization of RT-RPA-CRISPR assay conditions. CRISPR-FDS signal detected with RNA isolated from EVs purified from 50 μL plasma aliquots of individuals diagnosed with or without COVID-19 by positive or negative nasal RT-qPCR results.

FIG. 6. In silico validation. Specificity of CRISPR-ABC primers and gRNA using the indicated genomic viruses and virus RNA entries for SARS-CoV-2 isolates from different countries, other coronaviruses, and common respiratory viruses in FIG. 5. Symbols indicate primers judge able (+) or not able (−) to amplify or bind sequence derived from the indicated viruses using following criteria: at least 10 matching bases separated by no mismatches, no more than 5 total mismatches, and a Tm>50° C.

FIG. 7. Sources from viruses and viral RNA analyzed in FIG. 8.

FIG. 8. Optimization of RT-RPA-CRISPR assay conditions. CRISPR-FDS signal for RNA extracts obtained from healthy human plasma spiked (50 μL) with or without RNA or virions (>10⁵ copies) of the indicated human respiratory viruses. Data represent the mean±SD of three replicates.

FIG. 9. Schematic of the RT-RPA-CRISPR liposome synthesis workflow and reagents.

FIG. 10. Size distribution of RT-RPA-liposomes measured by NanoSight.

FIG. 11. Low-magnification TEM image of plasma-derived EVs used in the liposome-EV fusion reaction showing their size distribution and morphology. Scale bar: 200 nm.

FIG. 12. Size distribution of EVs with or without liposome fusion measured by NanoSight.

FIG. 13. Representative TEM images of RT-RPA-CRISPR liposomes at low and magnification.

FIG. 14. Representative TEM images of RT-RPA-CRISPR liposomes at high magnification

FIG. 15. Representative TEM images of liposome-EV fusion reactions at low magnification.

FIG. 16. Representative TEM images of liposome-EV fusion reactions at high magnification.

FIG. 17. Schematic of results from an assay measuring the increase in FRET donor signal (588 nm) and decrease in FRET acceptor signal (673 nm) due to dye separation on labeled EVs (2×108) as a result of increased distance following membrane fusion after incubation with 1× (2×108) or 10× (2×109) molar ratios of unlabeled empty liposomes.

FIG. 18. Results from an assay measuring the increase in FRET donor signal (588 nm) and decrease in FRET acceptor signal (673 nm) due to dye separation on labeled EVs (2×108) as a result of increased distance following membrane fusion after incubation with 1× (2×108) or 10× (2×109) molar ratios of unlabeled empty liposomes. Data represent the mean±SD of three replicates.

FIG. 19. CRISPR-FDS liposome assay signal detected with EVs isolated from 293F cells stably transfected with a SARS-CoV-2 N gene expression vector or the empty expression vector.

FIG. 20. Map of the lentiviral expression vector construct (pLenti-CMV-CoVN-His) that contains the full-length SARS-CoV-2 N gene.

FIG. 21. CRISPR-FDS assay analysis of EV RNA isolated from a 293F cell line that stably expresses the SARS-CoV-2 N gene from pLenti-CMV-CoVN-His expression vector, which refers to FIG. 3a. Data represent mean±SE of experimental triplicates.

FIG. 22. CRISPR-FDS liposome assay kinetics upon analysis of EVs captured from 50 μL plasma aliquots of individuals diagnosed with and without COVID-19 (EV pos/EV neg samples) upon incubation with CRISPR-FDS reagent-loaded liposomes with or without polyethylene glycol (PEG), or with free CRISPR-FDS reagents.

FIG. 23. CRISPR-FDS liposome assay kinetics detected upon analysis of plasma aliquots (50 μL) from an individual with COVID-19 diagnoses based on positive nasal swab RT-qPCR results. EVs from the subject with positive nasal swab results were at 90° C. for 30 min then incubated with reagent-loaded liposomes (Heat EVs+liposome), CRISPR-FDS reagents not packed into liposomes (Heat EVs+free CRISPR-FDS reagents), EVs without heating were incubated with reagent-loaded liposomes in the absence of PEG (liposome only) or CRISPR-FDS reagents not packed into liposomes (free CRISPR-FDS reagents).

FIG. 24. Relative EV abundance in plasma from COVID-19 patients and healthy donors, refers to FIG. 3d. EV ELISA signal obtained from EVs captured from 50 μL plasma with an antibody to the EV surface protein CD81 when captured EVs were sequentially incubated with biotin labeled anti-CD9 antibody, streptavidin-conjugated horseradish peroxidase, and the chromogenic dye tetramethylbenzidine, after which absorbance at 450 nm was measured with a plate-reader.

FIG. 25. CRISPR-FDS liposome assay signal detected for plasma aliquots from 20 adults with COVID-19 and 10 adults without COVID-19, as diagnosed by nasal swab RT-qPCR results.

FIG. 26. Demographics of the adult cohort analyzed in FIGS. 23-25.

FIG. 27. EV COVID-19 assay cross activity evaluation in lung diseases refer to FIGS. 23-25. Analysis of plasma aliquots (50 μL) from 5 individuals COVID-19 diagnoses based on positive and 5 negative nasal swab RT-qPCR results, 6 pulmonary tuberculosis (PTB) infection 5 Non-tuberculosis Mycobacterium (NTM), 19 pneumonia, 3 Cystic fibrosis (CF), and 1 Allergic Bronchopulmonary Aspergillosis (ABPA).

FIG. 28. CRISPR-FDS liposome assay signal detected for plasma samples from six patients diagnosed with COVID-19 based on (A40-A45) who were diagnosed COVID-19 negative by nasal swab sample and a healthy donor (HD) and positive control (PC, RT-qPCR positive nasal swab sample). Data represent the mean±SD of three replicates.

FIG. 29. Timeline for sample collection (plasma and nasal swabs) relative to SARS-CoV-2 infection in the African green monkey COVID-19 model.

FIG. 30. Demographics of lung disease cohort analyzed in FIG. 27.

FIG. 31. Normalized CRISPR-FDS liposome signal intensity from NHP plasma samples and RT-RPA-CRISPR signal intensity from NHP nasal swab samples at the indicated timepoints.

FIG. 32. Positive (red) and negative (blue) results for CRISPR-FDS liposome (EV) fluorescent intensity, nasal RT-qPCR, and serological results (IgG) in three children at the indicated time points after first evaluation. Data represent the mean±SD of three replicates.

FIG. 33. Proteomic analysis of plasma exosomes isolated from 3 COVID-19 patients and 3 healthy donors who were diagnosed by nasal swab RT-qPCR. Numbers indicated the number of EV proteins identified by mass spectrometry from healthy donors and COVID-19 patients, and their overlap when detected peptides were searched against the UniProtKB protein database. No SARS-CoV-2 proteins were identified in this search.

FIG. 34. Western blot analysis of SARS-CoV-2 N protein expression in protein lysates (50 μg/lane) of plasma EVs isolated from three COVID-19 patients (A1-A3). Positive control (PC), 10 μg SARS-CoV-2 recombinant protein; Negative control (NC), 50 μg EVs isolated from healthy donor plasma.

FIG. 35. Oligonucleotide list.

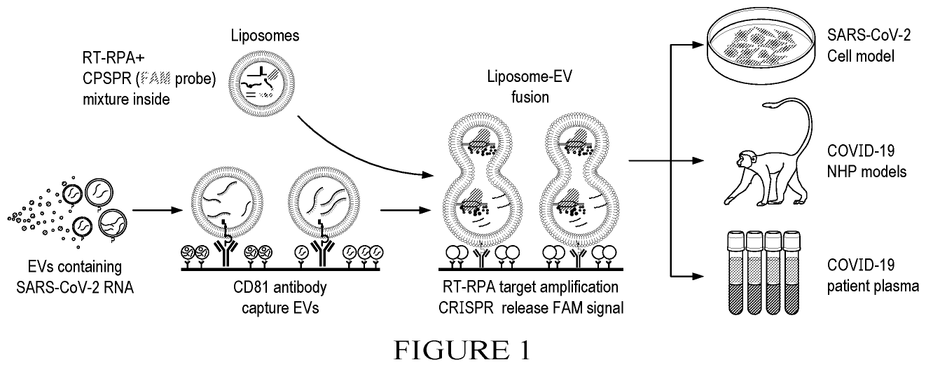

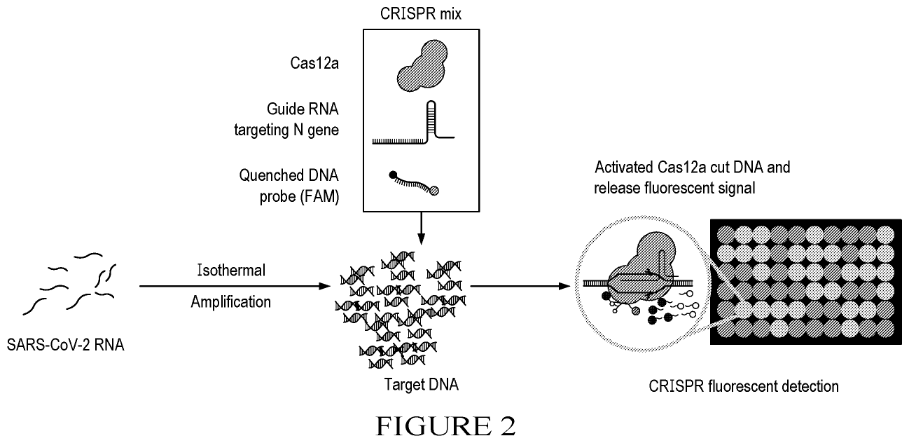

### DETAILED DESCRIPTION

The disclosure provides novel method and system for detecting disease presence in a blood sample and extracted EVs, as opposed to conventional testing method that requires respiratory RNA sample. Plasma SARS-CoV-2 RNA may represent a viable diagnostic alternative to respiratory RNA levels that rapidly decline after infection. RT-qPCR reference assays exhibit poor performance with plasma, likely reflecting dilution and degradation of viral RNA released into the circulation, but these issues could be addressed by analyzing viral RNA packaged into extracellular vesicles (EVs).

The disclosure also provides an assay approach where EVs directly captured from plasma are fused with reagent-loaded liposomes to sensitively amplify and detect a SARS-CoV-2 gene target. This approach accurately diagnosed COVID-19 patients, including challenging cases missed by RT-qPCR. SARS-CoV-2-positive EVs were detected at day one post-infection, and plateaued from day six to the day 28 endpoint in a non-human primate model, while 20-60 day signal durations were observed in young children. This nanotechnology approach addresses unmet needs for COVID-19 diagnosis by extending diagnosis windows and detecting missed cases with a non-infectious sample.

To achieve the results, the present disclosure describes a method for detecting the presence of disease-specific proteins in a bodily fluid sample, comprising the steps of: (a) extracting extracellular vesicles (EVs) in the bodily fluid sample by use of a first antibody against the disease-specific protein; (b) mixing liposome fusion probes with the EVs in step (a), wherein the liposome fusion probes contain RT, recombinase polymerase amplification (RPA), and CRISPR/Cas12a reagents; and (c) detecting the presence of the disease-specific protein using a fluorescent detection system.

The method and system of the present disclosure focuses on extracellular vesicles that in a subject have at least one disease protein. EVs have their specific surface markers that can be targeted by antibodies, whereas the at least one disease protein also have epitopes targeted by antibodies. As such, one can simultaneously detect both pathogenetic and host targets in body fluids that contain EVs.

The present disclosure describes a novel method of detecting the presence of disease in a sample by first extracting the extracellular vesicles (EVs) in the sample, followed by detecting the disease-specific markers from the EVs. To do this, the first step is to identify the disease-specific markers that are present in EVs, and can therefore be captured.

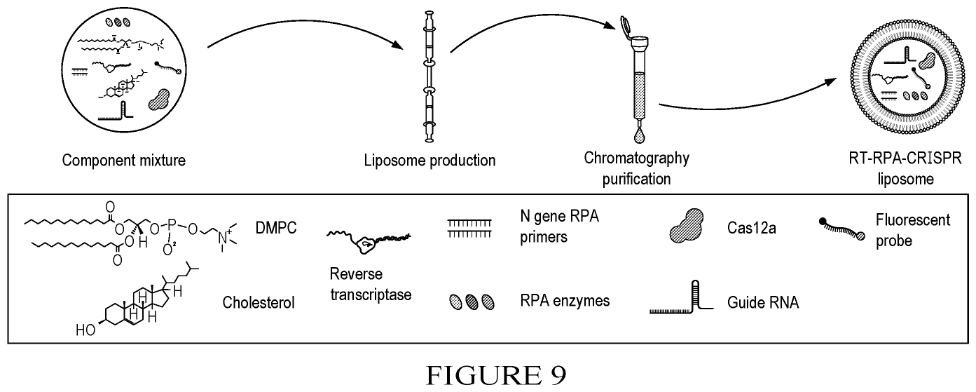

Nanoscale liposomes synthesized to deliver RT-RPA-CRISPR reagents to captured EVs (FIG. 9) exhibited uniform morphology (˜100 nm mean diameter;

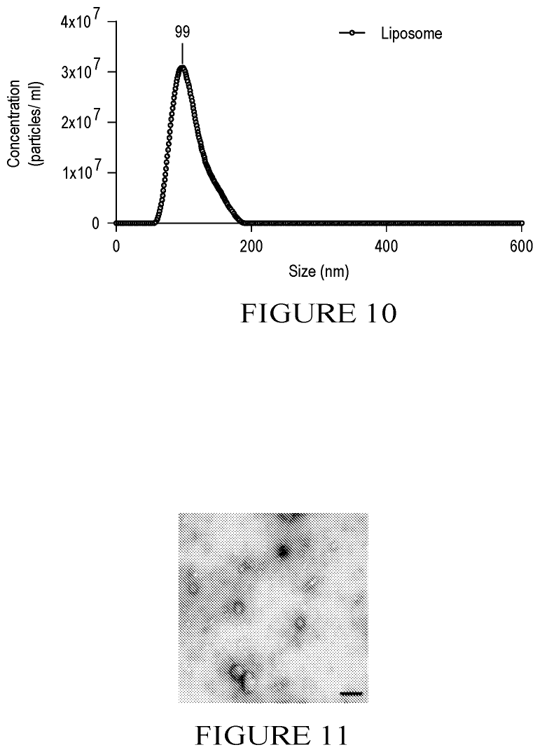

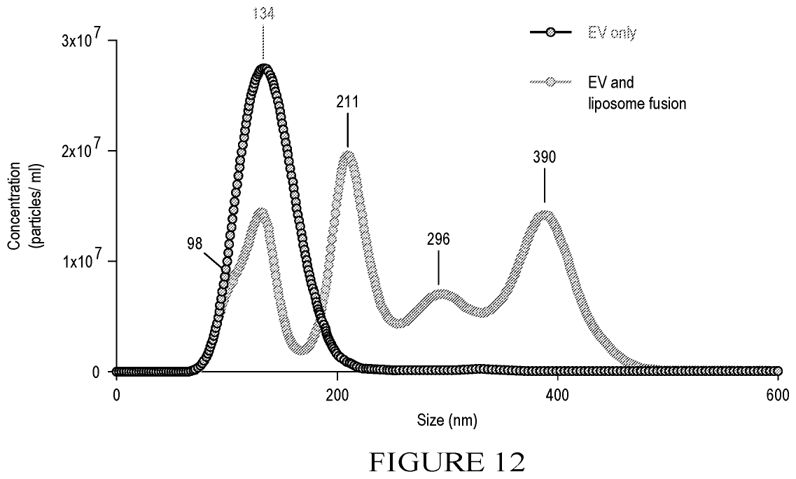

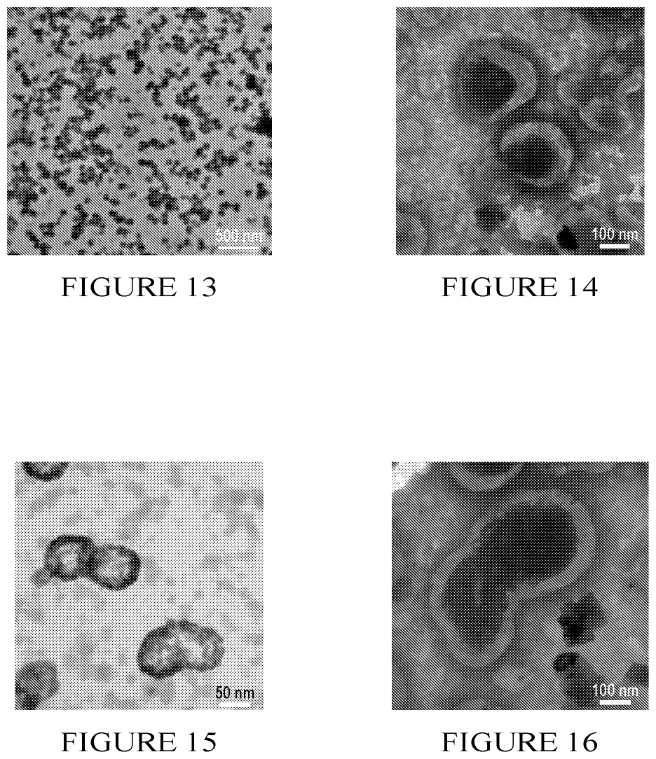

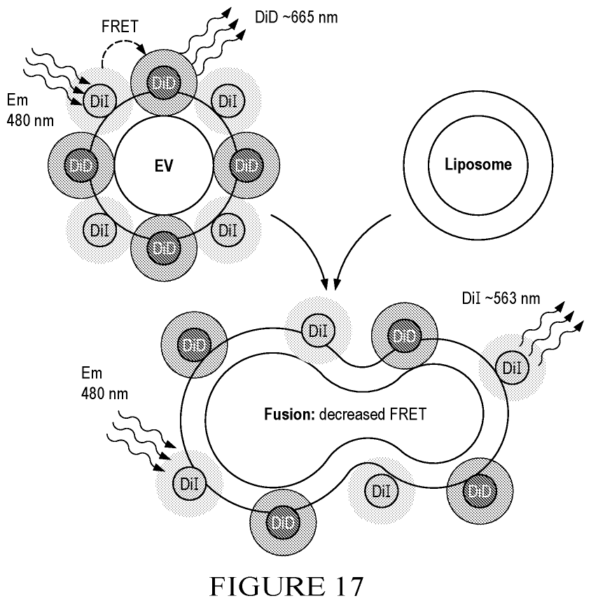

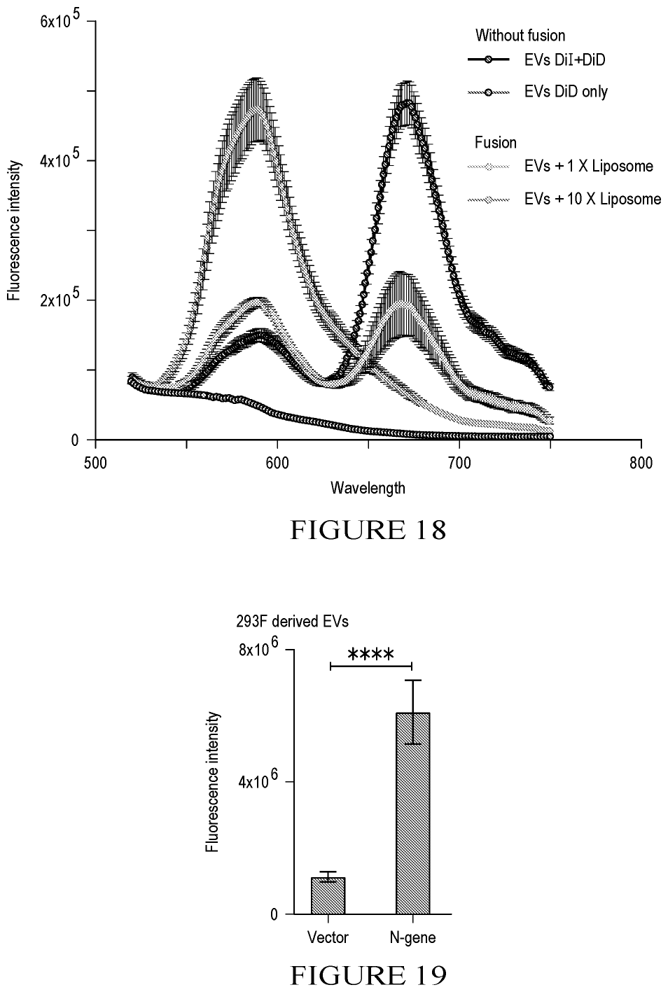

FIG. 10), and produced fusion products when incubated with purified EVs and PEG800019 (FIG. 11, 12). Transmission electron microscopy (TEM) analysis of these reactions (FIGS. 13-16) revealed 200 nm vesicles consistent with incomplete fusion products (FIGS. 15-16). To confirm the presence of EV-liposome fusion events, EVs were dual-labeled with 1,1′-dioctadecyl-3,3,3′,3′ tetramethylindocarbocyanine perchlorate (DiI; acceptor) and DiIC18(5) (DiD; donor) in a Förster resonant energy transfer (FRET) dequenching assay (FIG. 17). FRET activity decreased with the liposome-to-EV ratio, consistent with a liposome membrane contribution diluting the FRET dyes to respectively enhance and attenuate donor and acceptor signal (FIG. 18).

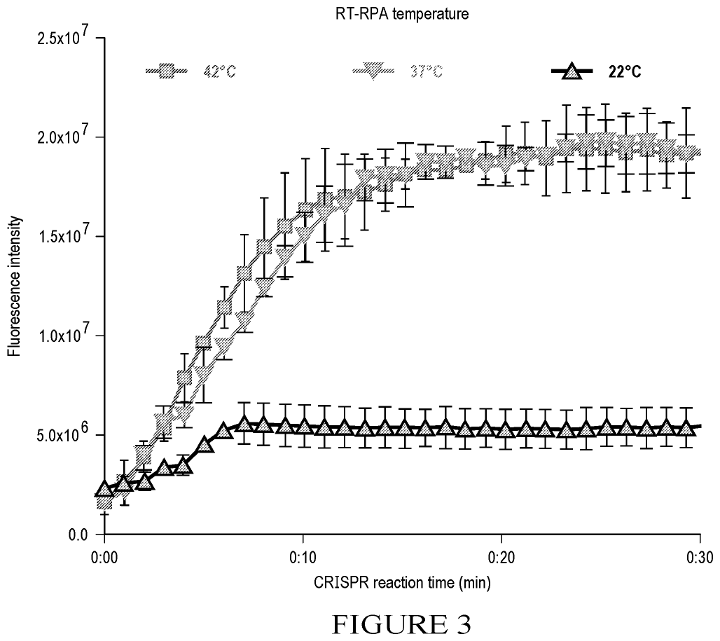

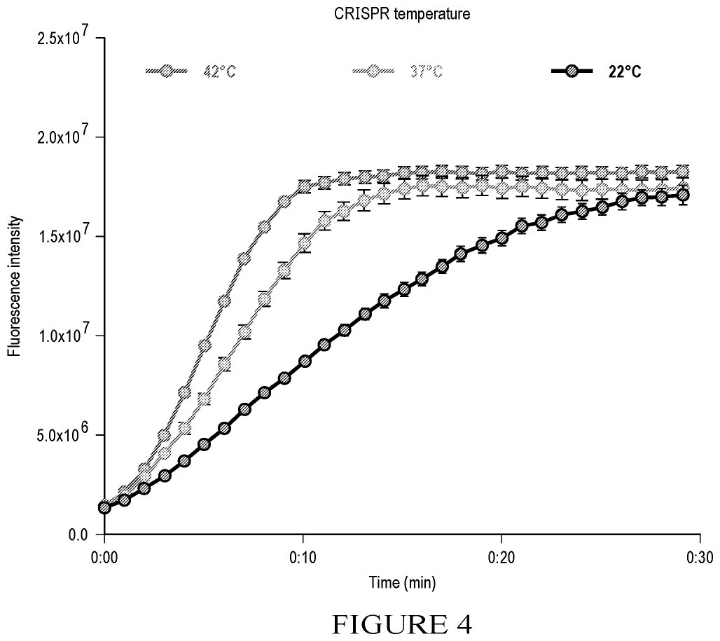

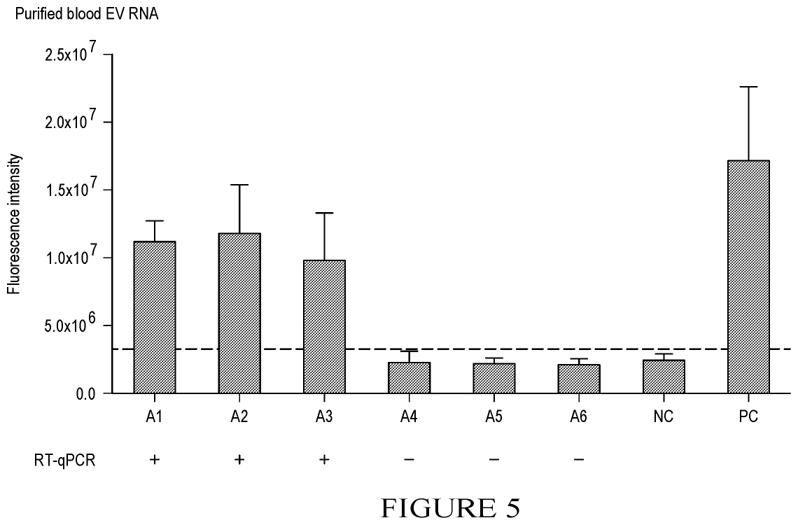

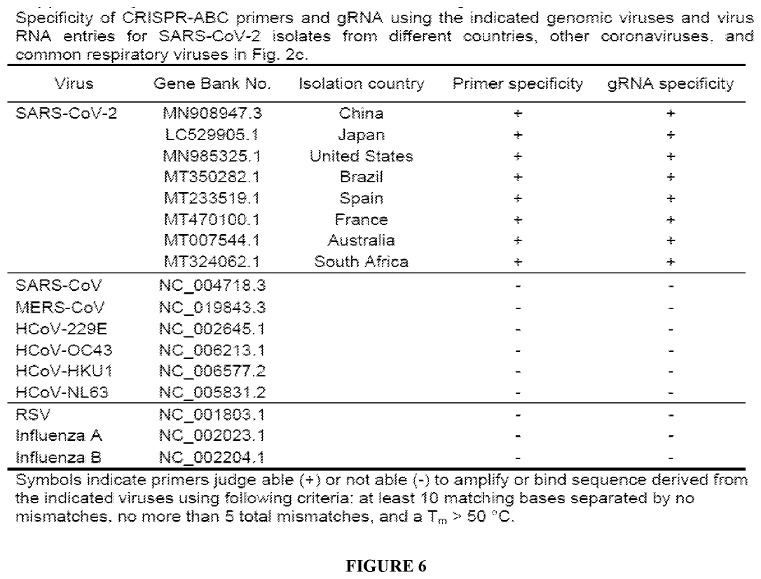

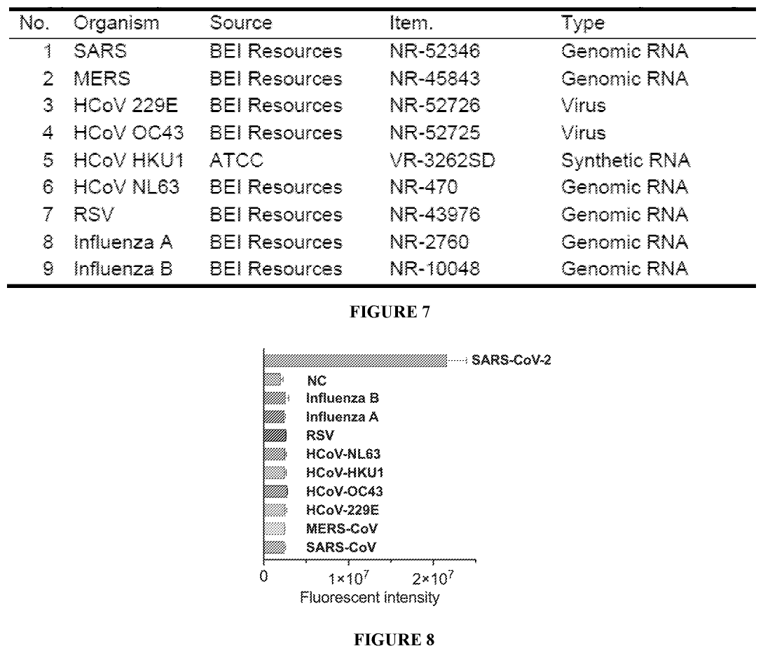

EV capture and EV-liposome fusion reactions occur over broad temperature ranges. We therefore optimized conditions for a one-step CRISPR-enhanced RT-RPA fluorescent detection system (CRISPR-FDS) suitable for incorporation into liposome fusion probes, using assay conditions from a previous study as our starting point. RT-RPA reactions performed at room temperature (˜22° C.) did not reveal marked CRISPR-mediated probe conversion under optimal CRISPR conditions, while RT-RPA reactions performed within the optimal RT-RPA temperature range produced similar CRISPR-FDS signal (FIG. 3). CRISPR reactions performed with a constant amount of amplicon demonstrated different reaction kinetics but similar endpoints, with reactions performed at 22° C., 37° C. and 42° C. reaching their respective maximum signal intensities at approximately 25, 13 and 10 minutes (FIG. 4). CRISPR-FDS assays using these optimized conditions to analyze plasma EVs RNA isolates accurately distinguished individuals with and without COVID-19 (FIG. 5). However, negative control samples containing genomic RNA from other human respiratory viruses, including influenza and several alpha and beta coronaviruses (FIG. 6, 7), produced no signal, confirming the SARS-CoV-2-specificity of this one-step assay approach (FIG. 8).

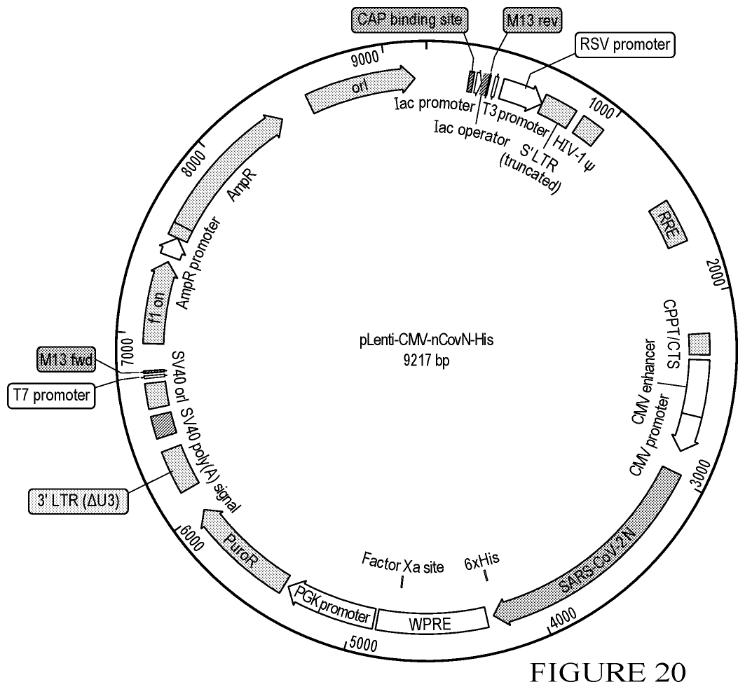

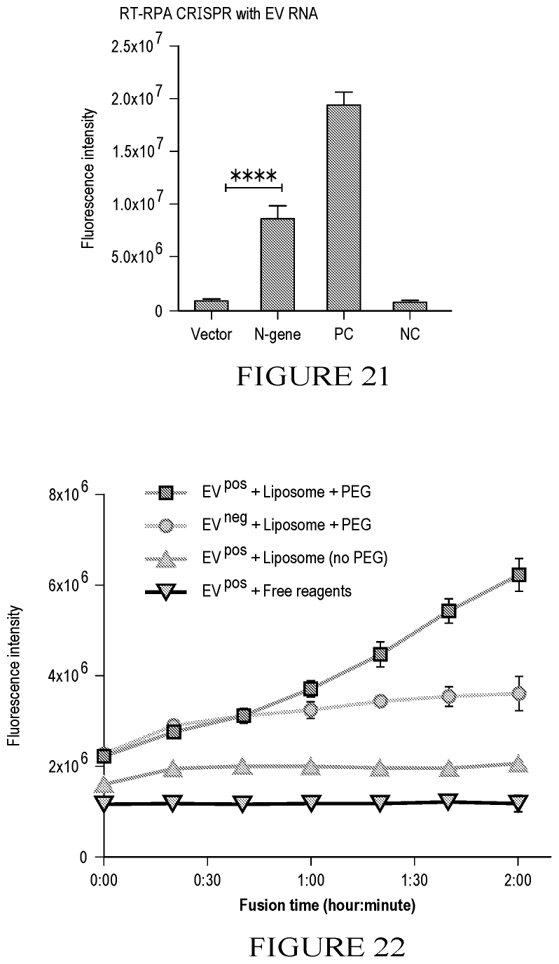

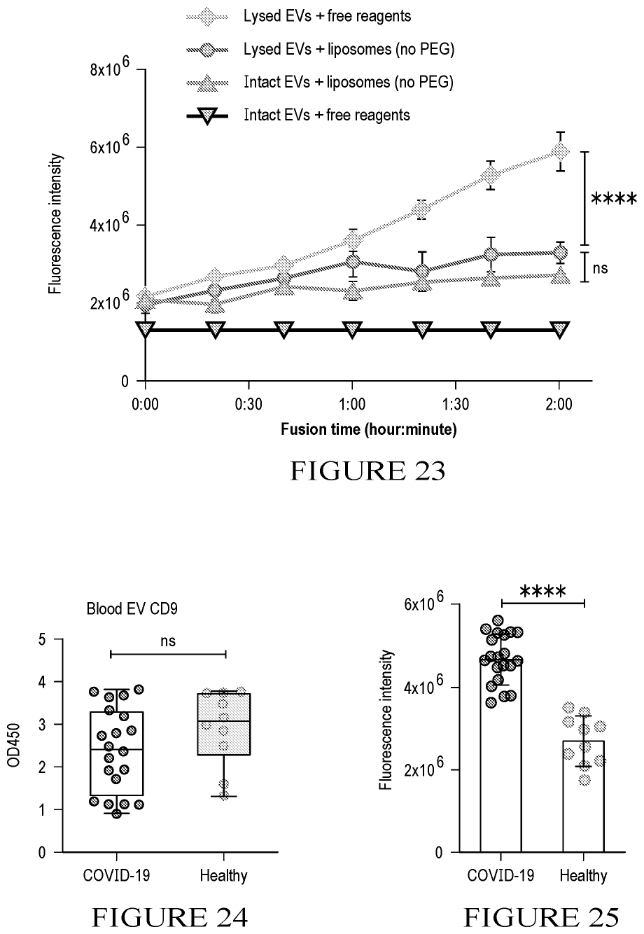

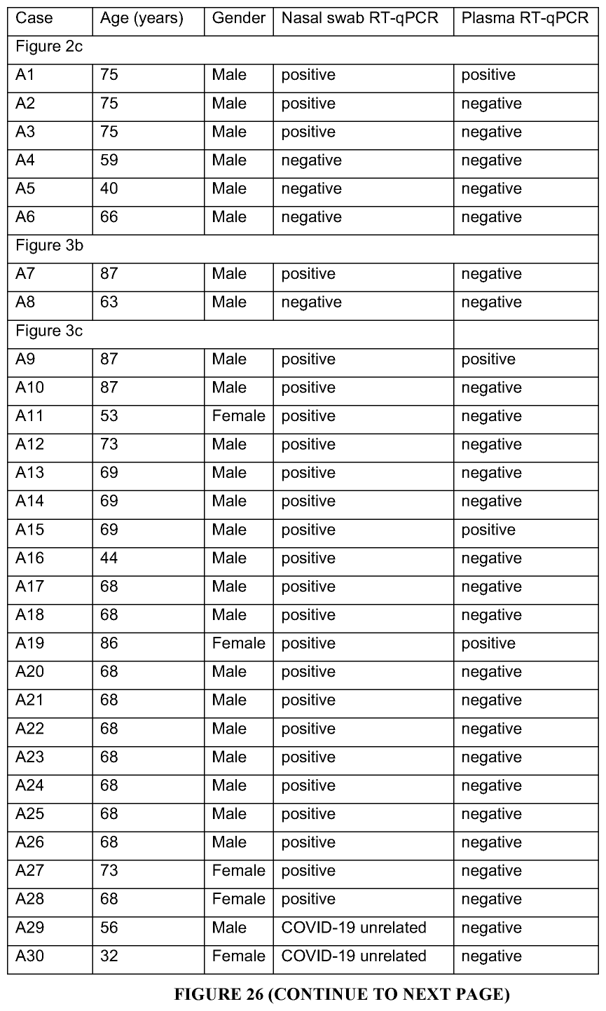

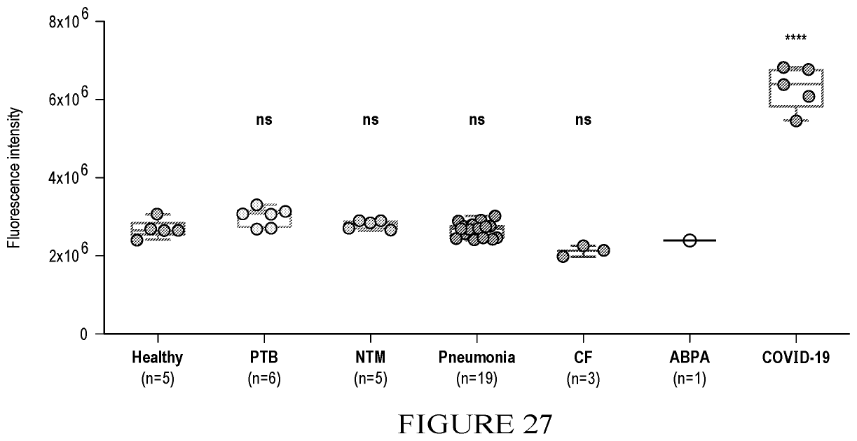

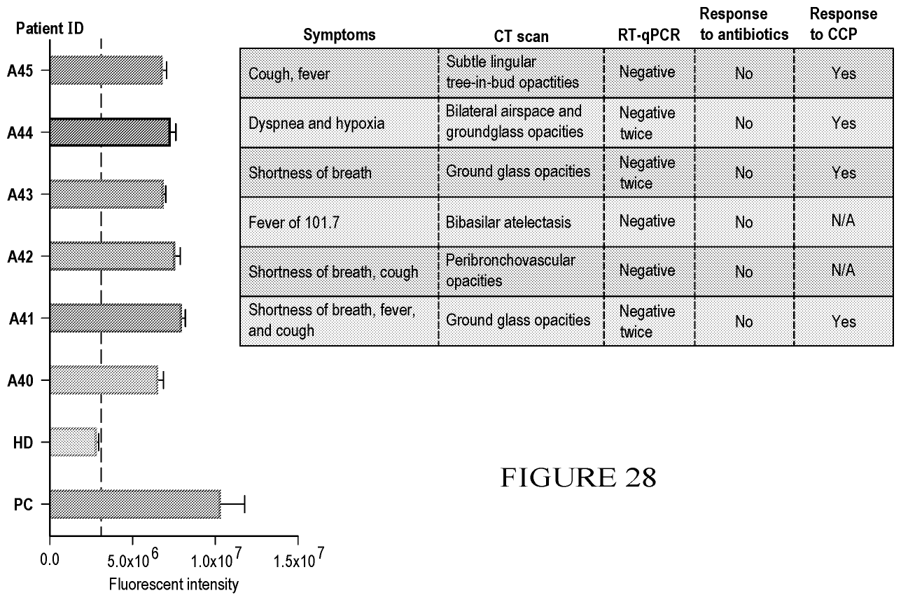

Liposomes loaded with the RT-RPA-CRISPR reagents were then incubated with antibody-captured EVs to evaluate their ability to detect EV RNA targets upon vesicle fusion. This analysis found that CRISPR-FDS signal detected with EVs captured from cells expressing the SARS-CoV-2 N gene was significantly greater than signal from EVs of control cells (FIG. 19-21). Plasma aliquots from three individuals with and without COVID-19 also revealed CRISPR-FDS signal differences one hour after initiating the EV-liposome fusion reaction, which steadily increased until the two hour reaction endpoint, while signal did not differ without fusion (FIG. 22, 23). Similar results were obtained with a larger patient cohort, where both groups exhibiting similar overall plasma EV levels (FIG. 24), but mean CRISPR-FDS intensity was >1.7× higher in individuals diagnosed with COVID-19 (FIG. 25, 26). No CRISPR-FDS cross-reactivity was observed upon analysis of plasma samples from patients with other lung diseases (FIG. 27). CRISPR-FDS liposome assays performed on archived plasma samples from six hospitalized individuals with suspected COVID-19, who had pulmonary CT scans consistent with COVID-19 pathology but negative nasal swab RT-qPCR results, found that all six patients had detectable SARS-CoV-2 RNA in their plasma EVs (FIG. 28). Four patients also received COVID-19 convalescent plasma and improved after its administration, while both patients who did not receive COVID-19 convalescent plasma had SARS-CoV-2 specific antibodies upon retrospective analysis, supporting the clinical judgement that all six cases had COVID-19.

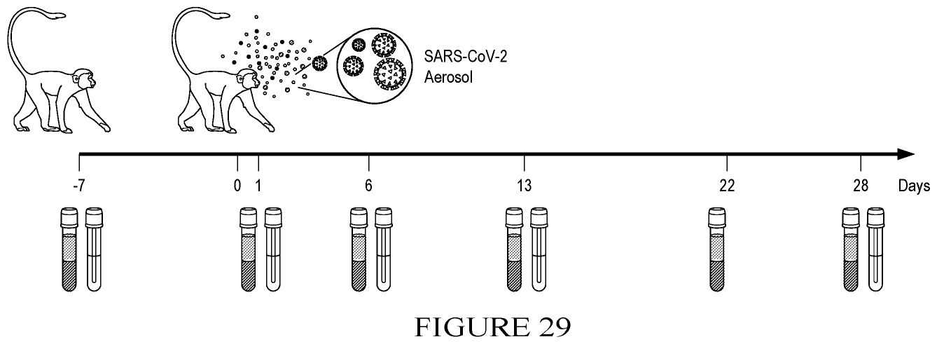

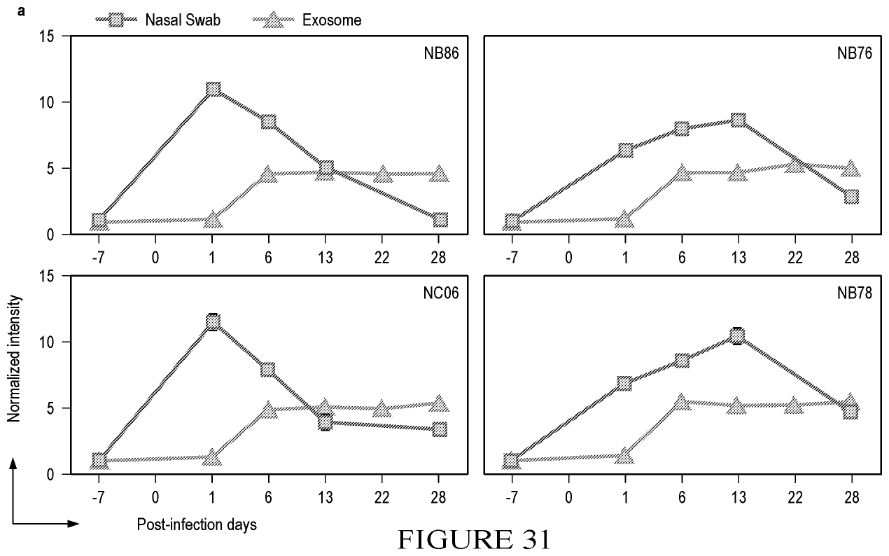

SARS-CoV-2 viral replication is detectable in the lower respiratory tract after it is undetectable in the upper respiratory tract, which may explain our detection of SARS-CoV-2 RNA in plasma EVs of patients with negative nasal swab results. To address potential changes in SARS-CoV-2 RNA level in nasal tissue and plasma EVs following infection, we analyzed serial samples collected from a non-human primate (NHP) model of SARS-CoV-2 infection in which adult African green monkeys were infected by low-dose SARS-CoV-2 aerosol exposure and then followed for 28 days (FIG. 29, 30). RT-qPCR analysis detected high nasal swab SAR-CoV-2 RNA levels at day 1 post-infection, which tended to peak between days 1 and 13 post-infection, and decrease rapidly after peak expression (FIG. 31). CRISPR-FDS liposome assay results for matching plasma samples, however, detected low plasma EV SARS-CoV-2 RNA levels at day 1 post-infection, which consistently increased at day 6 post-infection and remained stable for the entire time course, suggesting that EV SARS-CoV-2 RNA expression may be a more durable marker of infection.

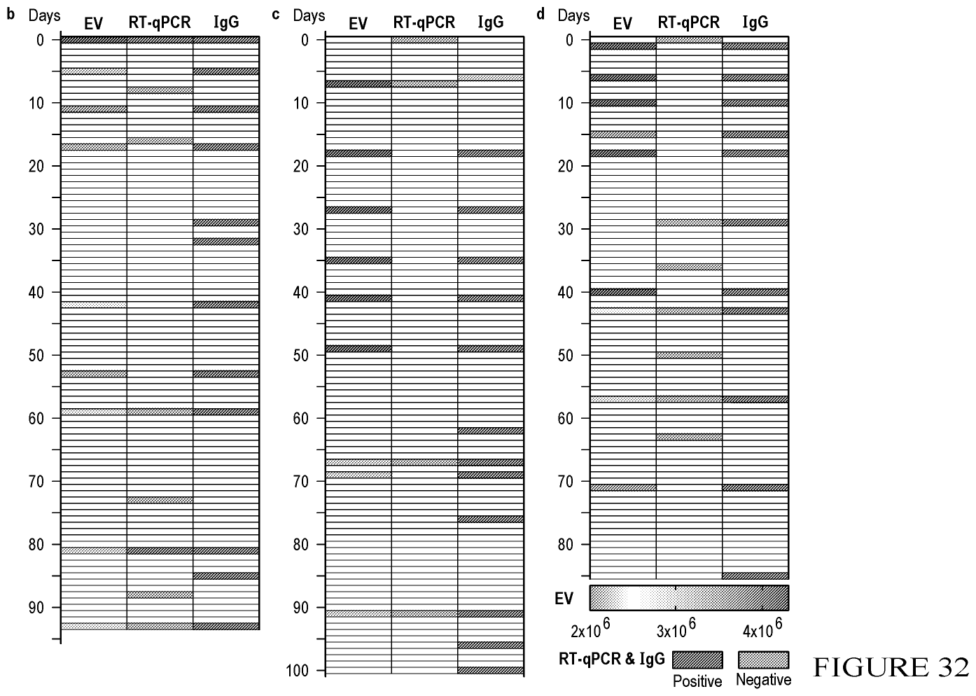

Since plasma EV SARS-CoV-2 RNA levels in this model exhibited a delayed and sustained peak relative to nasal swabs results, we examined the time course of SARS-CoV-2 EV signal in young children who demonstrated evidence of COVID-19 at or following initial evaluation and had available nasal swab RT-qPCR results and archived blood samples during a >3-month follow-up period. Two of these children were ≤1.5 years of age and had negative nasal swab results at all timepoints, but had SARS-CoV-2-positive plasma EV results from their initial evaluation to 40-60 days from this visit, with EV signal remaining positive ˜40 days after the first SARS-CoV-2 IgG positive sample (FIG. 32). The third child had a single positive RT-qPCR test followed by two negative tests, but four positive plasma EV results over the same interval, after which there was a two month period where both tests yielded negative results before again detecting SAR-CoV-2 RNA suggestive of disease recurrence or reinfection (FIG. 32). However, RT-qPCR detected a single positive sample followed by two negative samples, whereas all plasma samples collected within this interval were positive. SARS-CoV-2 IgG positive samples were detected throughout the evaluation period, but this child was only two months of age at first evaluation and thus detected IgG could have derived from the mother, whose infection status was unknown and who did not have samples available for analysis.

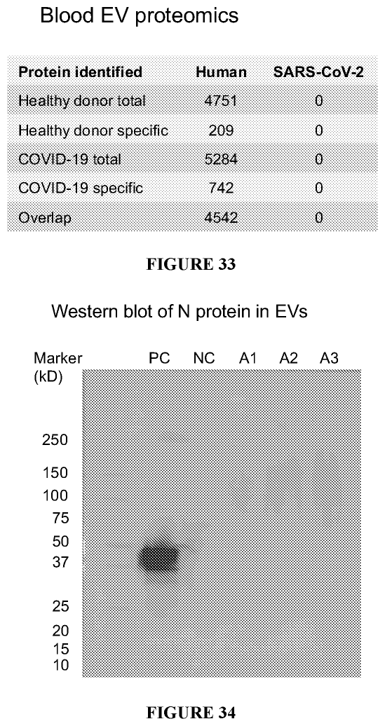

Our results indicate that EVs containing SARS-CoV-2 RNA are detectable in the circulation early after infection and persist after nasal swab RT-qPCR assays employed as the gold-standard for COVID-19 diagnosis return negative results. However, this study does not address several important questions. First, while our data indicate that SARS-CoV-2 RNA is detectable in plasma EVs from early infection onward, the format of EV RNA cargo is unclear. Proteomic analysis of plasma EVs isolated from COVID-19 patients did not detect SARS-CoV-2 viral protein (FIG. 33, 34), suggesting that EV cargoes containing SARS-CoV-2 RNA were not viral particles, although sensitivity was limited by the lack of a SARS-CoV-2 RNA-enriched EV sample. However, EV-derived SARS-CoV-2 RNA was detected under reaction conditions not expected to disrupt SARS-CoV-2 virions and release their RNA.

Second, the functional significance of detecting SAR-CoV-2 RNA in these EVs is unclear. Two other single strand plus-sense RNA viruses are reported to employ the EV biogenesis pathway to deliver their genomes to recipient cells to initiate infection, but it is not known if EVs found to carry SARS-CoV-2 RNA in the current study also support infection. Our CRISPR-FDS liposome assay detects a region of the SARS-CoV-2 N gene, but not whether EVs containing this region also contain the entire viral genome to support potential infection. Reports indicate that some COVID-19 patients develop SARS-CoV-2 infections at extrapulmonary sites implying that intact virus or EVs carrying SARS-CoV-2 RNA enter the circulation to initiate infections at secondary sites. SARS-CoV-2 RNA has been detected in the circulation of COVID-19 patients, but studies have yet to report isolation of virus activity from COVID-19 patient plasma or serum. Given that EVs can directly promote the systemic spread of other viral infections, similar studies should be conducted for SARS-CoV-2.

Most diagnostics analyze upper respiratory tract samples or saliva, where virus replication appears more transient than in lower respiratory tract, and potentially extrapulmonary, infections. We believe the major utility of our extraction-free approach is its ability to detect plasma EV-derived SARS-CoV-2 RNA as an early and durable sign of systemic infection. Our EV capture approach has several apparent advantages over alternate methods. For example, total plasma RNA isolates would contain more degraded RNA and off-target RNA and thus should have lower sensitivity and higher background. EV isolation and RNA extraction by standard methods would be laborious, time-consuming, and exhibit significant variation due to batch-to-batch differences in EV yield and purity. Several new approaches do not require viral RNA isolation or amplification, but these methods still require high viral loads, and analyze upper respiratory tract samples rather than blood. Our assay has potential utility as a secondary test for suspected COVID-19 cases that are RT-qPCR negative but lack alternative diagnoses. It may be particularly valuable for individuals with long-term evidence of infection since transient upper respiratory tract RT-qPCR results may not reflect virus levels in pulmonary or extrapulmonary infections. This includes individuals with compromised immune systems, such as transplant recipients and others receiving immunosuppressive therapies. It may also be relevant during organ donation to reduce the risk of virus transfer, as recently documented for a lung transplant case where the donor's SARS-CoV-2 infection was not detected by respiratory tract RT-qPCR testing and lead to the death of the recipient and infection of the surgeon. Our assay is intended as a clinical application since it analyzes plasma, requires wash steps, and utilizes a benchtop plate reader for its longitudinal readout. A portable device that utilizes a microfluid chip to generate and analyze a fingerstick blood sample, could potentially be developed for a point of care solution, although this would require stabilization of the reagent-loaded liposomes or an adaptation to analyze lysates of the captured EVs.

### Methods

### CRISPR-FDS Liposome Probe Synthesis

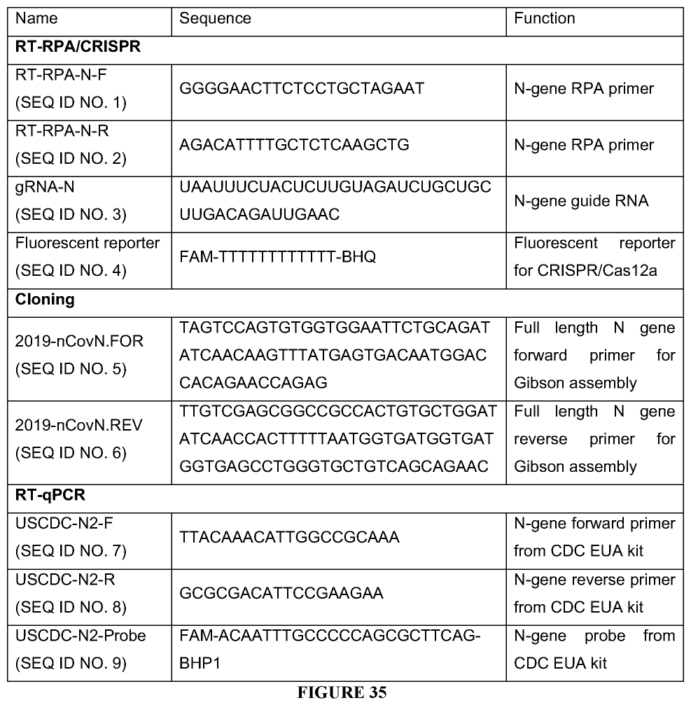

Nanoscale liposomes employed to deliver CRISPR-FDS reagents to EVs were synthesized by dissolving 48 μmol 1,2-Dimyristoyl-sn-glycerol-3-phosphorylcholine (DMPC) and 4.8 μmol cholesterol in 1 mL ethanol, which were mixed and dried under nitrogen gas. This material was then mixed with CRISPR-FDS reagents (10 μL RT enzyme, 300 μL 10× NEBuffer 2.1, 8.4 μmol MgOAC, 0.3 μmol N gene primer pairs, 10 tubes of TwistAmp™ Basic powder (TwistDx, UK), 300 μL rehydration buffer, 0.16 μmol Cas12a (NEB M0653T), 0.16 pmol N gene guide RNA, and 1 pmol FAM-labeled DNA probe (FIG. 35). CRISPR-FDS-loaded liposomes were then prepared by sequentially passing this lipid-reagent mixture through 0.8 μm, 0.4 μm, 0.2 μm and 0.1 μm polycarbonate membranes (20× for each filter) at room temperature, after which free reagents and lipid were removed by size exclusion chromatography using a G25 Dextran column. The liposome fraction was then analyzed by Nanosight to determine the liposome size distribution and diluted and vortexed in 5 mL PBS to generate a concentrated liposome solution (8.5×10⁹ liposomes/mL), which was aliquoted and stored at 4° C. until aliquots were diluted for use in CRISPR-FDS liposome assays.

### CRISPR-FDS Liposome Fusion Assay

Black-wall 96-well ELISA plates (Corning Costar 3370) coated with 1 μg anti-CD81 (Invitrogen, MD5-13548)/well by overnight incubation at room temperature were incubated with 100 μL purified EVs or plasma, as described, for 2 hours at 37° C. to allow EV capture, washed 3× with phosphate buffered saline with 0.05% Tween 20 (PBST). Sample wells were then incubated with 50 μL of a reaction solution containing 4.2×108 RT-RPA-CRISPR liposomes, adjusted to a final concentration of 25% mass/volume PEG8000, and incubated at 37° C. for 2 hours, after which CRISPR-FDS fluorescent signal was read with a benchtop plate reader (480 nm excitation; 525 nm emission). An EV assay result was considered positive if it was equal or greater than a cut-off threshold defined by the mean signal of the negative control samples plus three times their standard deviation.

EV isolation from cell culture medium. EV isolation from cell culture medium was performed as previously described. Briefly, ten 70-80% confluent 15 cm culture dishes of 293F cells (Invitrogen) were washed three times with PBS and then cultured in DMEM media supplemented with 10% EV-depleted FBS for 48 hours, after which conditioned media was collected and centrifuged at 2000 g for 30 minutes to remove cell debris and passed through a 0.45 m filter (LG-FPE4041505, LifeGene). EVs in this clarified supernatant were concentrated passing this material over a 100 KDa centrifugal filter unit (UFC901008, Thermo Fisher Scientific) at 3000 g for 20-30 minutes for three times. Retained sample was collected by washing the membrane 3× with 500 μL PBS, centrifuged twice at 4° C., 12,000 g for 30 minutes to precipitate residual debris. This supernatant was then centrifuged twice at 100,000 g and 4° C. for 3 hours, discarding the supernatant and resuspending the pellet in PBS after each centrifugation step. This EV fraction was then analyzed by Nanosight to determine EV size distribution and diluted and vortexed in 5 mL PBS to generate a concentrated EV solution (8.75×109 EVs/mL), which was aliquoted and stored at −80° C. until aliquots were diluted for use in CRISPR-FDS liposome assays.

Plasma EV isolation. Plasma EV samples used in FIG. 3b were isolated with an ExoQuick ULTRA EV Isolation Kit (EQULTRA-20A-1, System Biosciences) following manufacturer instructions. Briefly, 250 μL plasma aliquots were centrifuged at 3000 g for 15 minutes, supernatants were then gently mixed with 67 μL ExoQuick solution and incubated on ice for 30 minutes, and then centrifuged at 3000 g and 4° C., for 10 minutes. EV pellets were then processed according to the manufacturer's instructions, and EVs was then analyzed by NanoSight.

### Particle Size Measurement

The relative concentrations of purified plasma EV samples were measured by bicinchoninic acid (BCA) assay, and all samples were diluted to a 5 μg/mL final concentration in PBS before subsequent analysis. The size distributions and concentrations EVs and liposome samples were measured using a NanoSight NS300 instrument employing Nanoparticle Tracking Analysis Software (Malvern Instruments) and a capture duration of 60 s for each sample.

### TEM

Liposome or cell culture EV samples were diluted to a final concentration of ˜8.45×10⁹ vesicles/μL in 2% pH 7.0 phosphotungstic Acid (PTA), which plasma EVs were diluted to a final concentration of 50 ng EV protein/μL. Samples aliquots (20 μL) were then spotted on parafilm, and allowed to adhere for 20 minutes to a carbon-coated grid that was floated carbon side down over them, after which excess fluid was removed by wicking through filter paper. Grids were rinsed with distilled water before being placed carbon side down on a 20 μL drop of filtered 2% pH 7.0 PTA to stain for approximately 1 min, then the PTA was with filter paper wicking and samples were allowed to completely dry at room temperature. Images of liposome, EVs and vesicle fusions were captured using a FEI TECNAI F30 transmission electron microscope operating at 300 kV.

### FRET Analysis for Liposome-EV Fusion

EVs aliquots containing 2×108 EVs purified from human plasma samples were resuspended in 1 mL PBS containing 5 μL Vybrant DiI (Molecular Probes, V-22885) and 5 μL DiD (Molecular Probes, V-22887) and incubated at room temperature for 20 min, then filtered three times with a 100 kDa centrifugal filter unit (UFC901008, Thermo Fisher Scientific) at 3000 g for 20-30 minutes at room temperature to remove free dyes and concentrate EVs to a ˜50 μL final volume. Liposome aliquots containing 2×10⁸ or 2×10⁹ liposomes in 50 μL PBS were mixed with EVs double-labeled with DiI and DiD, and liposome-EV fusion reactions were performed as described above. Fluorescent signal was excited with 480 nm laser and fluorescent emission spectrum was measured with SpectraMax iD5 (Molecular Device) microplate reader from 525 nm to 750 nm.

### SARS-CoV-2 N Gene Expression Vector and Cell Line

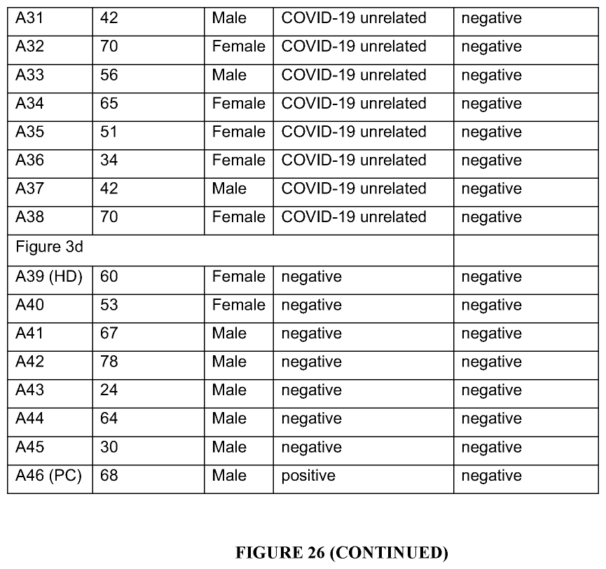

The SARS-CoV-2 N gene was PCR amplified using a 2019-nCoV_N_Positive Control (IDT 10006625) as the template, and then cloned into the pLenti-CMV-puro lentiviral vector (Addgene 17452) by Gibson assembly (FIG. 26). Candidate expression vector subclones expected to carry the N gene target region were validated for full-length sequence identity by Sanger sequencing. 293F cells (1×106) cells suspended in 2 mL were co-transfected with 2 μg of pLenti-CMV-puro expression vector with or without the N-gene subclone and 1.5 μg of the psPAX2 and 1 μg of the pMD2.G vectors. After 48 hours, 1 mL of conditioned culture media containing lentivirus from the transfected 293F cells was added to culture wells containing 0.5×106 HEK293F cells for 12 hours, after which cells were cultured with 1 μg/mL puromycin (Gibco A1113803) for 48 hours to select for transduced cells, which were collected and expanded in DMEM with 10% FBS to achieve cell cultures containing 3×108 cells for EV isolation, as described above.

### Clinical Sample Collection

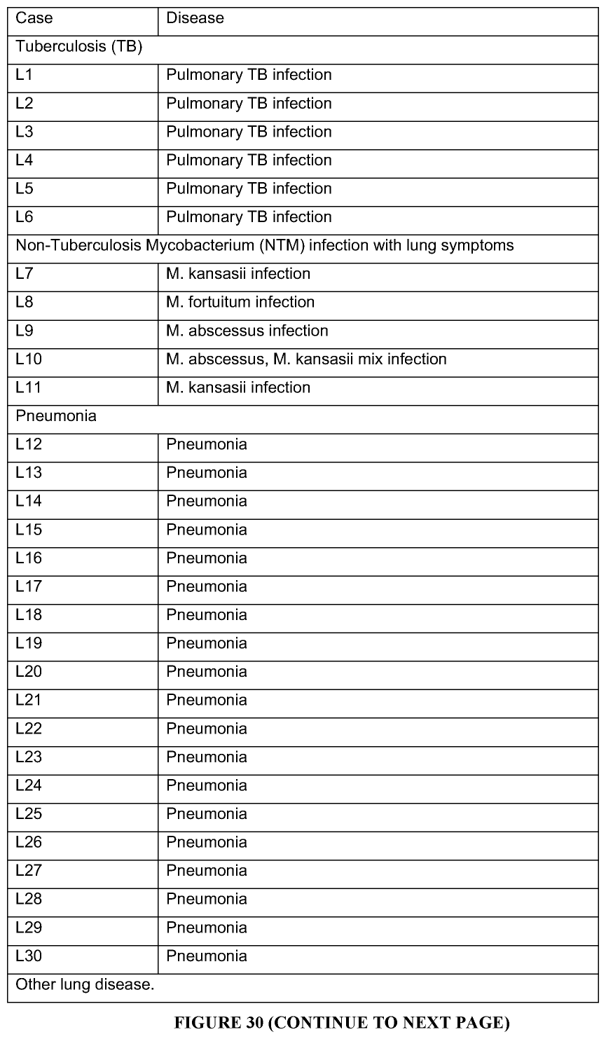

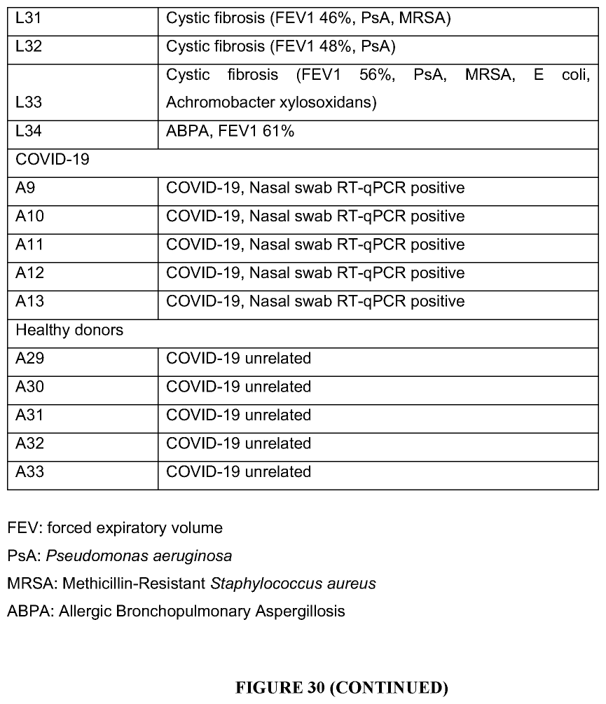

Human nasal swab and plasma specimens analyzed in this study and demographic data were collected after obtaining prior written informed consent from adult patients or the legal guardians of pediatric patients, who also indicated their assent, in compliance with approved IRB protocols. Samples analyzed in the adult cohort (FIG. 30) were obtained from patients who had matching plasma and nasal swab samples analyzed by the Tulane Molecular Pathology Laboratory between May 1 to Aug. 12, 2020, and whose COVID-19 status was determined based on clinical indications and current CDC guidance. Nasal swab results, demographic data and plasma samples from indicated cases was obtained from children who were screened for COVID-19 at regional children's hospital in Orleans Parish, Louisiana between March-Jul. 15, 2020 under a separate IRB (FIG. 30). Eligibility criteria included any child (≤18 years) receiving care at the children's hospital. Blood was drawn as part of care in the emergency room, inpatient floors, ambulatory clinics, or as part of routine pre-operative studies for time-sensitive surgeries. Plasma samples corresponding to the described adult case studies were obtained from individual who were treated at Tulane Medical Center between April 27 and Jul. 14, 2020, under a separate IRB protocol. Due to hospital regulations, refrigerated samples were release to our study team between three and seven days after blood draw. All identifying data was removed and samples were coded with a unique subject identification. Clinical results for nasal swab were determined using the CDC 2019-nCoV real-time RT-PCR Diagnostic Panel.

CCP treatment of adult case studies: Following written informed consent in accordance with the Declaration of Helsinki, ABO compatible CCP was infused over 1-2 hours following premedication with 650 mg of acetaminophen and 25 mg of diphenhydramine. One patient was treated after obtaining individual emergency Investigational New Drug (eIND) approval from the FDA (FIG. 28 patient) were enrolled in the investigator initiated clinical trial Expanded Access to Convalescent Plasma to Treat and Prevent Pulmonary Complications Associated With COVID-19.

Plasma and swab collection and processing procedures: Human and NHP blood samples were collected in EDTA tubes and rapidly processed to isolate plasma. NHP plasma samples were immediately stored at −80° C. until processed for RNA. Human plasma was obtained from the volume remaining in plasma stored at 4° C. for potential further clinical tests. Refrigerated adult and pediatric plasma samples refrigerated samples were released to our study team after 3-7 days and 7 days after blood draw, respectively. All identifying data was removed and samples were coded with a unique subject identification. Samples were then heat inactivated for 30 minutes at 56° C., and stored at −20° C. until processed for RNA. Human and NHP nasal swab samples and NHP rectal swab samples were collected in 200 μL of DNA/RNA Shield (R1200, Zymo Research) and stored at −80° C. until processed for RNA. NHP and clinical specimens were processed in an enhanced BL2/BL3 space in accordance with a protocol approved by the Institutional Biosafety Committee. RNA samples were isolated from 100 μL of plasma or swab storage buffer using the Zymo Quick-DNA/RNA Viral Kit (D7020) following the assay protocol, and RNA was eluted in 50 μL and stored at −80° C. until analysis.

COVID-19 IgG test: ELISA wells were antigen coated for 1 h at room temperature with 0.5 μg/ml purified SARS CoV-2 spike protein (kindly provided by Kathryn Hastie at Scripps Research Institute) suspended in fresh 0.1 M NaHCO₃. Wells were washed five times with PBS+EDTA and incubated with blocking buffer (PBS containing 0.5% Tween, 5% dry milk, 4% whey proteins, 10% FBS) for 30 min at 37° C. In parallel, a set of wells not coated with antigen were incubated with blocking buffer. Serum was heat inactivated, diluted 1:100 in blocking buffer, and 100 μL/well of diluted serum was incubated 1 h at room temperature. Wells were then washed and incubated for 30 min at room temperature with peroxidase-conjugated goat anti-human IgG-Fc (Jackson ImmunoResearch) diluted 1:5,000 in blocking buffer, PBS washed, and incubated with 100 μL/well Tetramethylbenzidine (TMB)-H2O2. Color development was stopped by the addition of 1M phosphoric acid, and optical density was read at 450 nm in a 96 well plate reader. For each sample, OD values observed with control wells were subtracted from OD values observed with S protein to calculate net OD. Samples will OD>0.4 were considered positive, based on a cut-off OD value established by preliminary screening of >50 pre-COVID19 human sera in which no false positives were detected.

Virus Information: SARS-CoV-2 isolate USA-WA1/2020 was acquired from BEI Resources, and the harvested stock determined to have a concentration of 1×106 TCID50/ml. The virus was passaged in VeroE6 cells in DMEM media with 2% FBS sequence confirmed by PCR and/or Sanger sequencing. Plaque assays were performed in Vero E6 cells.

Animals and Procedures: Samples from a total of four (4) nonhuman primates aged approximately 7.5 years (4 male Chlorocebus aethiops (African green monkeys)) were used for analysis in this in this study. For viral inoculation, animals were anesthetized and then acutely exposed by head-only inhalation to SARS-CoV-2 (WA12020) resulting in an individual inhaled dose of ˜2.5×103 TCID50. Animals were biosampled thereafter and observed for 28 days post-infection including twice-daily monitoring by veterinary staff.

Statistical analysis: CRISPR-FDS assay signal was expressed as the mean of ≥3 independent reactions±SD. GraphPad Prism 8 was used to calculate one-way ANOVA to determine the optimized condition of RT-RPA and calculate linear regression of the standard curve. Multiple group comparisons were conducted using one-way ANOVA. Differences were considered statistically significant at P<0.05.

Enzyme-linked immunosorbent assay (ELISA): EVs isolated from plasma samples of COVID-19 patients were assayed for CD9 expression by sandwich ELISA according to the following procedure, which was modified to measure EVs membrane protein. Briefly, 50 μL isolated serum EVs samples was applied onto the 96-well microplate, which was pre-coated with an anti CD81 murine monoclonal antibody (1:500, Invitrogen, MD5-13548) and subsequently blocked with 5% bovine serum albumin in PBST for 2 h at room temperature. After incubation at 4° C. overnight, wells were gently washed four times with PBST, and then incubated at room temperature for 2 h with 50 ng of anti CD9-biotin rabbit polyclonal antibody (1:2000, MA119485, Invitrogen) in 100 μL PBS. Wells were then washed 4 times with PBST 4 times, and incubated for 1 h at room temperature with 25 ng of streptavidin-conjugated horseradish peroxidase in 100 μL PBS. Wells were then incubated for at room temperature for 20 min substrate after the addition of 50 μL solution tetramethylbenzidine (TMB, 34022, Thermo Scientific), then supplemented with 50 μL of 2M H2SO4 solution to each well to terminate the reaction, and the absorbance at 450 nm was measured with a plate-reader (SpectraMax iD5, Molecular Device). Each sample was assayed with analytical runs that were performed in duplicate.

Standard curve LoQ, LoD, positive result cut-off threshold: A SARS-CoV-2 RNA standard curve was generated by serially diluting the SARS-CoV-2 RNA reference standard (1.05×105 RNA copies/μL) in liposomes to generate 0.2, 0.6, 1, 2, 20, 2×102, 2×103, 2×104 and 2×105 copy/μL standards. The limit of quantification (LoQ) was defined as LoQ=10×Sy/s, where Sy is the standard deviation of the zero standard and s is the slope of the calibration curve. The mean+3×SD of the fluorescent intensity of the adult healthy control samples was used to set the threshold for a positive sample results in plasma from individuals with suspected SARS-CoV-2 infections.

Label-free Quantitative proteomics Analysis: EVs isolated from COVID patients and healthy controls were lysed by sonication in lysis buffer for label-free quantitative proteomics analysis. Total protein was precipitated by the addition of pre-chilled (−20° C.) to the protein lysate at a 1:5 ratio of acetone to lysate, and incubated overnight at −20° C. Samples were then centrifuged at 10000 g and 4° C. for 30 minutes, after which protein pellets were resuspended in ammonium bicarbonate buffer, and then reduced with TCEP ((tris(2-carboxyethyl)phosphine), alkylated with iodoacetamide prior to overnight digestion with trypsin. Resulting peptide samples were then fractionated by stage-tips into 6 fractions. Proteomics analysis was performed on Thermo Q Exactive HF-X Hybrid Quadrupole-Orbitrap coupled with an Ultimate 3000 nano-LC and nanoelectrospray ionization. Peptides were separated with a nC18 analytical column (C18 Pepmap 100, 3 μm particle, 100 Å pore, 75 μm i.d.×150 mm) using 150 min buffer gradient a low flow rate at 300 nL/min. Data-dependent acquisition in positive mode was used for data collection. Acquired data was searched with Proteome Discoverer 2.4 using the SEQUEST search engine with label-free quantification workflow against the UniProt database of Homo sapiens and Severe acute respiratory syndrome coronavirus 2 (SARS-CoV-2). Search parameters utilized trypsin cleavage sites, with an allowance for 2 missed cleavage sites, and precursor and fragment mass tolerances of ±10 ppm and 0.6 Da. Carbamidomethyl of cysteine was set as a fixed modification, and oxidation of methionine as a variable modification.

Western blot for plasma EVs: Plasma EV protein lysates (50 μg/lane) were loaded onto two 4%-20% gradient SDS-PAGE gels (Bio-Rad) and transferred to nitrocellulose membranes (Bio-Rad) by using standard methods. Gels were blocked with 5% bovine serum albumin (BSA) in PBS with 0.05% Tween-20 (PBST). Then the membrane was incubated with a 1:1000 dilution of anti-SARS-CoV-2 Nucleocapsid (N) protein primary antibody (SinoBiological 40143-MM05) for 2 hours at room temperature and then incubated for 1 hour at room temperature with a 1:5000 dilution of goat anti-mouse-HRP secondary antibody, with 10 μg of recombinant SARS-CoV-2 N protein (SinoBiological 40588-V08B) added as positive control.

What is claimed is:

1. A method of detecting the presence of a disease-specific protein in a bodily fluid sample, comprising the steps of:
a) extracting extracellular vesicles (EVs) in the bodily fluid sample by use of a first antibody against the disease-specific protein;
b) mixing liposome fusion probes with the EVs in step a), wherein said liposome fusion probes contain RT, recombinase polymerase amplification (RPA), and CRISPR/Cas12a reagents; and
c) detecting the presence of the disease-specific protein using a fluorescent detection system.

2. The method of claim 1, wherein said disease-specific protein is selected from the group consisting of: CD81, PDCD6IP, HSPA8, ACTB, ANXA2, CD9, PKM, HSP90AA1, ENO1, ANXA5, HSP90AB1, CD63, YWHAZ, YWHAE, etc. as well as the antibody against LprG, LpqH, Alpha-crystallin (HspX), DnaK, GroEL2, KatG, SodA, and GlnA.

3. The method of claim 1, wherein in step a) the EVs are enriched by using a detecting antibody that is specific to an antigen selected from the group consisting of: CD4, CD8, CD9, CD19, CD20, CD57, CD91, CD63, PDCD6IP, HSPA8, PD-1, PD-L1, TSPN8, EGFR, HER2, KRAS, ACE2, TMEM119, ANXA2, ANXA5, HSP90AB1, YWHAZ, YWHAE, LprG, LpqH, LAM, Ag85B, EpCAM, EphA2, Alpha-crystallin (HspX), DnaK, GroEL2, KatG, SodA and GlnA, GP120, GP160, and GP40.

4. The method of claim 1, wherein the disease generating said disease-specific protein is cancer, including but not limited to lung cancer, breast cancer, pancreatic cancer, leukemia, or lymphoma.

5. The method of claim 1, wherein the disease generating said disease-specific protein is a viral infection, including but not limited to TB, HIV, or influenza.

6. The method of claim 1, wherein the disease generating said disease-specific protein is a bacterially induced disease.

7. The method of claim 1, wherein the disease generating said disease-specific protein is brain damage or neuron degeneration.

8. The method of claim 1, wherein the bodily fluid is blood, serum, sputum, urine, saliva, or other available bodily fluid

9. A diagnostic kit for detecting the presence of a disease-specific protein in a bodily fluid sample, comprising:
a) a first antibody against the disease-specific protein;
b) liposome fusion probes, wherein said liposome fusion probes contain RT, recombinase polymerase amplification (RPA), and CRISPR/Cas12a reagents; and
c) a fluorescent detection system.

10. The diagnostic kit of claim 9, wherein the disease specific protein is selected from the group consisting of: CD81, PDCD6IP, HSPA8, ACTB, ANXA2, CD9, PKM, HSP90AA1, ENO1, ANXA5, HSP90AB1, CD63, YWHAZ, YWHAE, etc. as well as the antibody against LprG, LpqH, Alpha-crystallin (HspX), DnaK, GroEL2, KatG, SodA, and GlnA.

11. The diagnostic kit of claim 9, further comprising a detecting antibody for extracting and enriching extracellular vesicles (EVs) in the bodily fluid sample, wherein detecting antibody is specific to an antigen selected from the group consisting of: CD4, CD8, CD9, CD19, CD20, CD57, CD91, CD63, PDCD6IP, HSPA8, PD-1, PD-L1, TSPN8, EGFR, HER2, KRAS, ACE2, TMEM119, ANXA2, ANXA5, HSP90AB1, YWHAZ, YWHAE, LprG, LpqH, LAM, Ag85B, EpCAM, EphA2, Alpha-crystallin (HspX), DnaK, GroEL2, KatG, SodA and GlnA, GP120, GP160, and GP40.

12. The diagnostic kit of claim 9, wherein the disease generating said disease-specific protein is cancer, including but not limited to lung cancer, breast cancer, pancreatic cancer, leukemia, or lymphoma.

13. The diagnostic kit of claim 9, wherein the disease generating said disease-specific protein is a viral infection, including but not limited to TB, HIV, or influenza.

14. The diagnostic kit of claim 9, wherein the disease generating said disease-specific protein is a bacterially induced disease.

15. The diagnostic kit of claim 9, wherein the disease generating said disease-specific protein is brain damage or neuron degeneration.

16. The diagnostic kit of claim 9, wherein the bodily fluid is blood, serum, sputum, urine, saliva, or other available bodily fluid


SEQUENCE LISTING (9 sequences)

SEQ ID NO: 1 - DNA, 22 bp, Artificial Sequence
  N-gene RPA primer-forward
ggggaacttctcctgctagaat

SEQ ID NO: 2 - DNA, 21 bp, Artificial Sequence
  N-gene RPA primer-reverse
agacattttgctctcaagctg

SEQ ID NO: 3 - RNA, 41 nt, Artificial Sequence
  N-gene guide RNA
uaauuucuacucuuguagaucugcugcuugacagauugaac

SEQ ID NO: 4 - protein, 18 aa, Artificial Sequence
  Fluorescent report for CRISPR/Cas12a
FAMTTTTTTTTTTTTBHQ

SEQ ID NO: 5 - DNA, 69 bp, Artificial Sequence
  Full length N gene forward primer for Gibson assembly
tagtccagtgtggtggaattctgcagatatcaacaagtttatgagtgacaatggaccacagaaccagag

SEQ ID NO: 6 - DNA, 84 bp, Artificial Sequence
  Full length N gene reverse primer for Gibson assembly
ttgtcgagcggccgccactgtgctggatatcaaccactttttaatggtgatggtgatggtgagcctgggtgctgtcagcagaac

SEQ ID NO: 7 - DNA, 20 bp, Artificial Sequence
  N-gene forward primer from CDC EUA kit
ttacaaacattggccgcaaa

SEQ ID NO: 8 - DNA, 18 bp, Artificial Sequence
  N-gene reverse primer from CDC EUA kit
gcgcgacattccgaagaa

SEQ ID NO: 9 - DNA, 23 bp, Artificial Sequence
  N-gene probe from CDC EUA kit (the 5′-FAM and 3′-BHP1 are skipped)
accaatttgccccagcgcttcag